(19) United States
(12) Patent Application Publication
(10) Pub. No.: US 2026/0250397 A1
(43) Pub. Date: Aug. 27, 2026

(54) TREATMENT OF GASTRIC CANCER

(71) Applicant: AMGEN INC., Thousand Oaks, CA (US)

(72) Inventors: Alissa KEEGAN, Santa Monica, CA (US); Zhezhen LI, Thousand Oaks, CA (US); Di ZHOU, Thousand Oaks, CA (US)

(73) Assignee: AMGEN INC., Thousand Oaks, CA (US)

(21) Appl. No.: 18/853,059

(22) PCT Filed: Apr. 4, 2023
(86) PCT No.: PCT/US2023/065298

§ 371 Date: Sep. 30, 2024

Related U.S. Application Data
(60) Provisional application No. 63/327,666, filed on Apr. 5, 2022.
(60) Provisional application No. 63/491,969, filed on Mar. 24, 2023.

(51) Int. Cl. C07K 16/28; A61K 31/513; A61K 31/555; A61K 31/7068; A61K 39/00; A61P 35/00

(52) U.S. Cl. CPC C07K 16/2863; A61K 31/513; A61K 31/555; A61K 31/7068; A61P 35/00; C07K 16/2818; C07K 16/2827; A61K 2039/507; A61K 2039/545; C07K 2317/24; C07K 2317/41; C07K 2317/565; C07K 2317/732; C07K 2317/76; C07K 2317/94

(57) ABSTRACT

Methods of treating gastric cancer or gastroesophageal junction cancer in subjects are described. The methods may comprise administering an anti-FGFR2b antibody to the subject. The methods may further comprise administering a chemotherapy regimen comprising (a) oxaliplatin and capecitabine; or (b) oxaliplatin and S-1 to the subject.

### FIELD

Embodiments herein relate to uses of antibodies against fibroblast growth factor 2 (FGFR2), including antibodies against the FGFR2 isoform FGFR2-IIIb (also referred to as FGFR2b), in the treatment of gastric cancer, such as gastric adenocarcinoma and gastroesophageal junction adenocarcinoma.

### INCORPORATION-BY-REFERENCE OF MATERIAL SUBMITTED ELECTRONICALLY

Incorporated by reference in its entirety herein is a nucleotide/amino acid sequence listing submitted concurrently herewith and identified as follows: One 13,841 byte XML document named “10130-WO01-SEC.xml,” created on Mar. 27, 2023.

### CROSS-REFERENCE TO RELATED APPLICATIONS

This application claims the benefit of U.S. Provisional Application No. 63/327,666, filed Apr. 5, 2022 and U.S. Provisional Application No. 63/491,969, filed Mar. 24, 2023, the disclosures of which are incorporated by reference herein.

### BACKGROUND

Gastric cancer (GC) represents the fifth most common cancer worldwide with over 1 million new cases globally each year (Bray et al, 2018). The highest incidence (>20 per 100,000 in men) is seen in China, Japan, Latin America, and Eastern Europe, whereas the lowest incidence (<10 per 100,000 in men) is seen in North America, parts of Africa, and Northern Europe. Only 27% of newly diagnosed gastric cancers are localized and advanced disease carries a dismal prognosis and treatment remains challenging with a 5-year overall survival (OS) rate <5% with less clear benefit (hazard ratio [HR]=0.94 [95% CI: 0.78 to 1.14]) for the patients with tumors with a Programmed death-receptor 1 (PD-L1) combined positive score (CPS) of <5. Thus, despite decreasing incidence, gastric cancer remains a serious health burden globally with high mortality rate.

Fibroblast growth factor receptor 2b (FGFR2b) is present on a substantial subset of gastric cancers (GCs) and is a target for new therapies that may improve treatment outcomes. The fibroblast growth factor (FGF) family members bind to four known tyrosine kinase receptors, fibroblast growth factor receptors 1-4 (FGFR1 4) and their isoforms, with the various FGFs binding the different FGFRs to varying extents (Zhang et al., J. Biol. Chem. 281:15694, 2006). A protein sequence of human FGFR2 is provided in, e.g., GenBank Locus AF487553. Each FGFR consists of an extracellular domain (ECD) comprising three immunoglobulin (Ig)-like domains (D1, D2 and D3), a single transmembrane helix, and an intracellular catalytic kinase domain (Mohammadi et al., Cytokine Growth Factor Revs, 16:107, 2005). FGFs bind to the receptors primarily through regions in D2 and D3 of the receptors. There is a contiguous stretch of acidic amino acids in the linker between D1 and D2 called the “acid box” (AB). The region containing D1 and AB is believed to be involved in autoinhibition of the receptor, which is relieved by binding to ligand.

The FGFRs are characterized by multiple alternative splicing of their mRNAs, leading to a variety of isoforms (Ornitz et al., J. Biol. Chem. 271:15292, 1996; see also Swiss-Prot P21802 and isoforms P21802-1 to -20 for sequences of FGFR2 and its isoforms). Notably, there are forms containing all three Ig domains (α isoform) or only the two Ig domains D2 and D3 domains without D1 (β isoform). In FGFR1, FGFR2, and FGFR3, all forms contain the first half of D3 denoted IIIa, but two alternative exons can be utilized for the second half of D3, leading to IIIb and IIIc forms. For FGFR2, these are respectively denoted FGFR2-IIIb and FGFR2-IIIc (or just FGFR2b and FGFR2c, respectively); the corresponding beta forms are denoted FGFR2(beta)IIIb and FGFR2(beta)IIIc. The FGFR2-IIIb form of FGFR2 (also denoted K-sam-II) is a high affinity receptor for both FGF1 and KGF family members (FGF7, FGF10, and FGF22) whereas FGFR2-IIIc (also denoted K-sam-I) binds both FGF1 and FGF2 well but does not bind the KGF family members (Miki et al., Proc. Natl. Acad. Sci. USA 89:246, 1992). Indeed, FGFR2-IIIb is the only receptor for KGF family members (Ornitz et al., 1996, supra) and is therefore also designated KGFR.

The FGFRs and their isoforms are differentially expressed in various tissues. FGFR2-IIIb (and the IIIb forms of FGFR1 and FGFR3) is expressed in epithelial tissues, while FGFR2-IIIc is expressed in mesenchymal tissues (Duan et al., J. Biol. Chem. 267:16076, 1992; Ornitz et al., 1996, supra). Certain of the FGF ligands of these receptors have an opposite pattern of expression. Thus, KGF subfamily members, including FGF7 (KGF), FGF10, and FGF22, bind only to FGFR2-IIIb (Zhang et al., supra) and are expressed in mesenchymal tissues, and so may be paracrine effectors of epithelial cells (Ornitz et al., 1996, supra). In contrast, the FGF4 subfamily members FGF4-6 bind to FGFR2-IIIc and are expressed in both epithelial and mesenchymal lineages, and so may have either autocrine or paracrine functions. Because of the expression patterns of the isoforms of FGFR2 and their ligands, FGFR2 plays a role in epithelial-mesenchymal interactions (Finch et al., Dev. Dyn. 203:223, 1995), so it is not surprising that knock-out of FGFR2-IIIb in mice leads to severe embryonic defects and lethality (De Moerlooze et al., Development 127:483, 2000).

### SUMMARY

The disclosure provides a method of treating gastric cancer in a subject, which comprises administering to the subject an anti-FGFR2b antibody; and administering to the subject a chemotherapy regimen comprising: (a) oxaliplatin and capecitabine; or (b) oxaliplatin and S-1.

In some embodiments of the above method, the anti-FGFR2b antibody is administered Q3W at a dose of at least 15 mg/kg.

In some embodiments of the above method, the anti-FGFR2b antibody is administered Q3W at a dose of 15-22 mg/kg, or greater than 15 mg/kg to no more than 22 mg/kg.

In some embodiments, the above method further comprises administering an additional dose of the anti-FGFR2b antibody of 5-15 mg/kg 7-10 days after a first administration of the Q3W administration of the anti-FGFR2b antibody.

In some embodiments of the above method, the additional dose of the anti-FGFR2b antibody is 6-9 mg/kg or 7-8 mg/kg.

In some embodiments of the above method, the anti-FGFR2b antibody is administered Q3W at a dose of at least 20 mg/kg.

In some embodiments of the above method, the anti-FGFR2b antibody is administered Q3W at a dose of 20-30 mg/kg, or 22-25 mg/kg, or greater than 22 mg/kg to no more than 30 mg/kg, or greater than 22 mg/kg to no more than 25 mg/kg.

In some embodiments, the above method further comprises administering an additional dose of the anti-FGFR2b antibody of 10-15 mg/kg 7-10 days after a first administration of the Q3W administration of the anti-FGFR2b antibody.

In some embodiments of the above method, the additional dose of the anti-FGFR2b antibody is 9-13 mg/kg or 10-12 mg/kg.

In some embodiments of the above method, the anti-FGFR2b antibody is administered intravenously.

In some embodiments, the above method further comprises administering the oxaliplatin intravenously at a dose of at least 65 mg/m² Q3W and administering the capecitabine orally at a dose of at least 500 mg/m² twice a day.

In some embodiments of the above method, the oxaliplatin is administered intravenously at a dose of 100-150 mg/m² Q3W, and the capecitabine is administered orally at a dose of 750-1500 mg/m² twice a day.

In some embodiments, the method further comprises administering the oxaliplatin intravenously at a dose of at least 65 mg/m² Q3W, and administering the S-1 orally at a dose of at least 20 mg/m² twice a day.

In some embodiments of the above method, the oxaliplatin is administered intravenously at a dose of 100-150 mg/m² Q3W, and the Si is administered orally at a dose of 30-60 mg/m² twice a day.

In some embodiments of the above method, the anti-FGFR2b antibody is administered intravenously Q3W at a dose of 15-22 mg/kg, and is further administered at an additional dose of 7-12 mg/kg 7-10 days after a first administration of the anti-FGFR2b antibody Q3W, and administering the chemotherapy regimen comprises: (a) administering the oxaliplatin intravenously at a dose of at 100-150 mg/m² Q3W and administering the capecitabine orally at a dose of 750-1500 mg/m² twice a day; or (b) administering the oxaliplatin intravenously at a dose of at 10-150 mg/m² Q3W and administering the S-1 orally at a dose of 30-60 mg/m² twice a day.

In some embodiments of the above method, the anti-FGFR2b antibody is administered intravenously Q3W at a dose of 15-22 mg/kg, and is further administered at an additional dose of 7-12 mg/kg 7-10 days after a first administration of the anti-FGFR2b antibody in the Q3W regimen, and administering the chemotherapy regimen comprises: (a) administering the oxaliplatin intravenously at a dose of at 130 mg/m² Q3W and administering the capecitabine orally at a dose of 1000 mg/m² twice a day; or (b) administering the oxaliplatin intravenously at a dose of at 130 mg/m² Q3W and administering the S-1 administered orally at a dose of 40 mg/m² twice a day.

In some embodiments of the above method, the anti-FGFR2b antibody is administered intravenously Q3W at a dose of 22-30 mg/kg, and is further administered at an additional dose of 10-12 mg/kg 7-10 days after a first administration of the anti-FGFR2b antibody Q3W, and administering the chemotherapy regimen comprises: (a) administering the oxaliplatin intravenously at a dose of at 130 mg/m² Q3W and administering the capecitabine orally at a dose of 1000 mg/m² twice a day; or (b) administering the oxaliplatin intravenously at a dose of at 130 mg/m² Q3W and administering the S-1 orally at a dose of 40 mg/m² twice a day.

In some embodiments, the above method further comprises administering an anti-PD-1 or anti PD-L1 antibody to the subject.

In some embodiments of the above method, the anti-PD-1 or anti PD-L1 antibody is administered Q3W.

In some embodiments of the above method, the anti-PD-1 antibody comprises nivolumab, pembrolizumab, orcemiplimab, or the anti-PD-L1 antibody comprises atezolizumab, avelumab, or durvalumab.

In some embodiments, the above method further comprises administering nivolumab to the subject at a dose of at least 300 mg Q3W.

In some embodiments of the above method, the nivolumab is administered at a dose of 300 mg-400 mg Q3W.

In some embodiments of the above method, the nivolumab is administered at a dose of 360 mg Q3W.

In some embodiments of the above method, the anti-FGFR2b antibody comprises a heavy chain variable region comprising a heavy chain complementarity determining region (HCDR) 1 of SEQ ID NO: 6, a HCDR2 of SEQ ID NO: 7, and a HCDR3 of SEQ ID NO: 8; and a light chain variable region comprising a light chain complementarity determining region (LCDR) 1 of SEQ ID NO: 9, a LCDR2 of SEQ ID NO: 10, and a LCDR3 of SEQ ID NO: 11.

In some embodiments of the above method, the anti-FGFR2b antibody is afucosylated.

In some embodiments of the above method, the heavy chain variable region of the anti-FGFR2b antibody comprises an amino acid sequence at least 95% identical to SEQ ID NO: 4, and the light chain variable region of the anti-FGFR2b antibody comprises an amino acid sequence at least 95% identical to SEQ ID NO: 5.

In some embodiments of the above method, the heavy chain variable region of the anti-FGFR2b antibody comprises the amino acid sequence of SEQ ID NO: 4, and the light chain variable region of the anti-FGFR2b antibody comprises the amino acid sequence of SEQ ID NO: 5.

In some embodiments of the above method, the anti-FGFR2b antibody comprises the heavy chain of SEQ ID NO: 1, and the light chain of SEQ ID NO: 2.

In some embodiments of the above method, the anti-FGFR2b antibody is bemarituzumab.

In some embodiments of the above method, cells of the gastric cancer overexpress FGFR2b.

In some embodiments of the above method, the cells of the gastric cancer overexpress FGFR2b mRNA or protein, or comprise an FGFR2 gene amplification.

In some embodiments of the above method, cells of the gastric cancer overexpress FGFR2b as determined by immunohistochemistry (IHC).

In some embodiments of the above method, at least 5%, 10%, 15%, or 20% of cells of the gastric cancer express FGFR2b as determined by IHC.

In some embodiments of the above method, the cells of the gastric cancer have a FGFR2b staining intensity of at least 2+, or 3+ as determinized by IHC, such as at least 5%, 10%, 15%, or 20% of cells of the gastric cancer having said FGFR2b staining intensity.

In some embodiments of the above method, the gastric cancer of the subject overexpresses FGFR2b, as determined by FGFR2b staining intensity of at least 2+, or 3+ in any cells of a sample of the gastric cancer, as determinized by IHC.

In some embodiments of the above method, the gastric cancer of the subject overexpresses FGFR2b, as determined by FGFR2b staining intensity of at least 2+, or 3+ in at least 10% of cells of a sample of the gastric cancer, as determinized by IHC.

In some embodiments of the above method, the gastric cancer comprises gastric cancer or gastroesophageal junction cancer.

In some embodiments of the above method, the chemotherapy regimen comprises (a) oxaliplatin and capecitabine.

### BRIEF DESCRIPTION OF THE DRAWING(S)

FIG. 1 is a diagram of methods of treating gastric cancer.

FIGS. 2A-B are diagrams of amino acid sequences. FIG. 2A depicts amino acid sequences of anti-FGFR2b antibodies of some embodiments. FIG. 2B depicts amino acid sequences of FGFR2's of some embodiments.

### DETAILED DESCRIPTION

Described herein are methods of treating gastric cancer in a subject. The cells of the gastric cancer may overexpress FGFR2 isoform FGFR2-IIIb (also known as FGFR2b). The methods can comprise administering an anti-FGFR2b antibody such as bemarituzumab to the subject. The methods can further comprise administering a chemotherapy regimen, such as oxaliplatin and capecitabine (CAPOX) or oxaliplatin and S-1 (SOX). The anti-FGFR2b antibody may be administered with the CAPOX or SOX in the same composition, or may be administered in separate compositions (sequentially or simultaneously). It is contemplated herein that the dosing of the anti-FGFR2b antibody monotherapy may be aligned with CAPOX and SOX regimens (typically once every three weeks (Q3W)). For example, the anti-FGFR2b antibody may be administered to the subject Q3W at a dose of at least 15 mg/kg, such as 15-22 mg/kg, greater than 15 mg/kg to no more than 22 mg/kg, 20-30 mg/kg, 22-25 mg/kg, greater than 22 mg/kg to no more than 30 mg/kg, or greater than 22 mg/kg to no more than 25 mg/kg. For example, the anti-FGFR2b antibody may be administered to the subject Q3W at a dose of at least 15 mg/kg or at least 20 mg/kg. In some embodiments, the methods may further comprise administering to the subject an additional dose of the anti-FGFR2b antibody of about 5-15 mg/kg 7-10 days after a first administration of the Q3W administration of the anti-FGFR2b antibody. For example, the additional dose of the anti-FGFR2b antibody may be about 10-15 mg/kg (e.g., 10 mg/kg, 11 mg/kg, 12 mg/kg, 13 mg/kg, 14 mg/kg, or 15 mg/kg) administered 7-10 days after a first administration of the Q3W administration of the anti-FGFR2b antibody. For example, the anti-FGFR2b antibody may be administered Q3W to the subject in a first administration at a dose of at least 15 mg/kg (e.g., 15-22 mg/kg) and an additional dose of the anti-FGFR2b antibody of 5-15 mg/kg (e.g., 6-9 mg/kg, 7-8 mg/kg, 9-13 mg/kg, or 10-12 mg/kg) 7-10 days after the first administration of the anti-FGFR2b antibody.

The administration schedule of the anti-FGFR2b antibody may be synchronized with the administration schedule of the oxaliplatin and capecitabine (CAPOX) or oxaliplatin and S-1 (SOX), minimizing the number of administration sessions for the subject.

Fibroblast growth factor receptor 2b (FGFR2b) is present on a substantial subset of gastric cancers. Though results vary significantly based on the assay used and the stage of the tumor, up to 61% of patients with gastric cancer have been reported to have FGFR2b overexpressing tumors (Ahn et al, 2016; Tokunaga et al, 2016; Han et al, 2015). Fibroblast growth factor receptor (FGFR) signaling plays an important role in cell growth and survival (Katoh and Katoh, 2006).

A recent study evaluating the pathological and prognostic significance of FGFR2 overexpression in patients with gastric cancer, including 4294 patients from 10 studies, found that FGFR2-overexpressing gastric cancer was associated with a significantly worse survival compared with tumors showing low FGFR2 expression (HR=1.40, 95% CI. 1.25 to 1.58; p<0.00001) (Kim et al, 2019), suggesting that inhibition of FGFR2b may be an important therapeutic target (Jung et al, 2012; Matsumoto et al, 2012).

Chemotherapy for advanced gastric prolongs survival and improves symptoms (Waddell et al, 2014; Kang et al, 2009; Okines et al, 2009; Al-Batran et al, 2008; Wagner et al, 2006). The combination of a platinum agent with a fluoropyrimidine has become a frequently used combination (Kang et al, 2009) and in a recent meta-analysis has been identified as superior to single agent treatment and best supportive care (Wagner et al, 2006). Although there is no single standard globally-accepted first line reference chemotherapeutic regimen for advanced gastric cancer, the combination of a fluoropyrimidine (5-FU, capecitabine or S-1) and a platinum agent (cisplatin or oxaliplatin) is an accepted standard of care in both Western and Asian countries (Kang et al, 2009; Okines et al, 2009; Al-Batran et al, 2008; Keam et al, 2008; Wagner et al, 2006).

### Anti-FGFR2b Antibodies

As used herein, “antigen binding protein” has its customary and ordinary meaning as understood by one of ordinary skill in the art in view of this disclosure. It refers to a protein that specifically binds a specified antigen. The term encompasses intact antibodies as well as derivatives, variants, fragments, and mutants thereof. An antigen binding protein also includes bivalent and polyvalent/multivalent constructs as well as bispecific and polyspecific/multispecific constructs, as well as domain antibodies, scFvs, and both membrane-bound and soluble receptors. In some embodiments, an antigen binding protein comprises, consists essentially of, or consists of an antibody. In any of the methods described herein, an anti-FGFR2b antigen binding protein may be administered to the subject. By way of example, the antigen binding protein may comprise or consist of an antibody, for example bemarituzumab.

An antibody is an example of an antigen binding protein. As used herein, “antibody” has its customary and ordinary meaning as understood by one of ordinary skill in the art in view of this disclosure. It refers to an immunoglobulin of any isotype with specific binding to the target antigen, and includes, for instance, chimeric, humanized, fully human, and monoclonal antibodies. An “antibody” as such is a subgenus of an antigen binding protein. For example, human or humanized antibodies can be of any isotype, including IgG (including IgG1, IgG2, IgG3 and IgG4 subtypes), IgA (including IgAQ1 and IgA2 subtypes), IgM and IgE. A human IgG antibody generally will comprise two full-length heavy chains and two full-length light chains. Antibodies may be derived solely from a single source, or may be “chimeric,” that is, different portions of the antibody may be derived from two or more different antibodies from the same or different species. It will be understood that once an antibody is obtained from a source, it may undergo further engineering, for example to enhance stability and folding. Accordingly, it will be understood that a “human” antibody may be obtained from a source, and may undergo further engineering, for example in the Fc region. The engineered antibody may still be referred to as a type of human antibody. Similarly, variants of a human antibody, for example those that have undergone affinity maturation, will also be understood to be “human antibodies” unless stated otherwise. In some embodiments, the antigen binding protein comprises, consists essentially of, or consists of a human, humanized, or chimeric monoclonal antibody.

A “heavy chain” of an antigen binding protein (such as an antibody) includes a variable region (“VHI”), and three constant regions: CH1, CH2, and CH3. A “light chain” of an antigen binding protein (such as an antibody) includes a variable region (“VL”), and a constant region (“CL”). Human light chains include kappa chains and lambda chains.

“Antigen binding region” means a protein, or a portion of a protein, that specifically binds a specified antigen. For example, that portion of an antigen binding protein that contains the amino acid residues that interact with an antigen and confer on the antigen binding protein its specificity and affinity for the antigen is referred to as “antigen binding region.” An antigen binding region typically includes one or more “complementary binding regions” (“CDRs”) of an antibody. A “CDR” is an amino acid sequence that contributes to antigen binding specificity and affinity. Antigen binding regions of antibody heavy and light chains generally exhibit the same overall structure, comprising relatively conserved framework regions (FR) joined by three CDRs. The CDRs from the two chains of each heavy chain/light chain pair typically are aligned by the framework regions to form a structure that binds specifically with a specific epitope on the target protein. From N-terminal to C-terminal, naturally-occurring light and heavy chain variable regions both typically conform with the following order of these elements: FR1, CDR1, FR2, CDR2, FR3, CDR3 and FR4. A numbering system has been devised for assigning numbers to amino acids that occupy positions in each of these domains. This numbering system is defined in Kabat Sequences of Proteins of Immunological Interest (1987 and 1991, National Institutes of Health, Bethesda, Md.), or Chothia & Lesk, 1987, J. Mol. Biol. 196: 901-917; Chothia et al., 1989, Nature 342: 878-883. In some embodiments, the CDRs of an antigen binding protein are defined according to the definition of Kabat or Chothia.

Antigen binding proteins against FGFR2b, such as antibodies, may be used in methods described herein. The antibodies may specifically bind to FGFR2b. In the method of some embodiments, the anti-FGFR2b antigen binding protein binds with a higher affinity to FGFR2b than to FGFR2-IIIc. For example, the anti-FGFR2b antibodies may not detectably bind to FGFR-IIIc. In the method of some embodiments, anti-FGFR2b antigen binding protein (e.g., antibody) binds to FGFR2b and blocks or inhibits signaling through the FGFR2b receptor. For example, the binding of the anti-FGFR2b antigen binding protein (e.g., antibody) to FGFR2b may inhibit phosphorylation of FGFR2 or a MAP kinase downstream of FGFR2. In the method of some embodiments, the anti-FGFR2b antigen binding protein (e.g., antibody), upon binding to FGFR2b, inhibits binding between FGFR2b and an FGF ligand thereof, such as FGF1 and/or FGF2.

Binding of antigen binding protein (e.g., antibody) to FGFR2b and inhibition of binding between FGFR2b and FGFs can be assessed, for example, by ELISA assays, as described in U.S. Pat. No. 8,101,723, or, for example, by a chip-based assay as described in Example 2 of WO 2015/017600. In some embodiments, the antibody induces an ADCC activity, and in some embodiments possesses enhanced ADCC activity, for example, as described in WO 2015/017600. ADCC activity, for example, may be determined as described in Example 3 of WO 2015/07600. In some embodiments, the antibody may inhibit growth of a human tumor in a mouse model, for example, as shown in Example 1 of WO 2017/091577. In some embodiments, the anti-FGFR2-IIIb antibody is capable of increasing the number of one or more of PD-L1 positive cells, NK cells, CD3+ T cells, CD4+ T cells, CD8+ T cells, and macrophages in tumor tissue in a mouse tumor model compared to a control, for example, as described in Example 2 of International Application No. WO 2017/091577.

Any of the anti-FGFR2b antibodies described herein may be afucosylated. For example, the antibody may be an IgG1 or IgG3 antibody that lacks fucose at Asn297. As used herein, an “afucosylated” antibody or an antibody “lacking fucose” refers to an IgG1 or IgG3 isotype antibody that lacks fucose in its constant region glycosylation. Glycosylation of human IgG1 or IgG3 occurs at Asn297 (N297; EU number of Fc region residue) as core fucosylated biantennary complex oligosaccharide glycosylation terminated with up to 2 Gal residues. In some embodiments, an afucosylated antibody lacks fucose at Asn297. These structures are designated as G0, G1 (α1,6 or α1,3) or G2 glycan residues, depending on the amount of terminal Gal residues. See, e.g., Raju, T. S., BioProcess Int. 1: 44-53 (2003). CHO type glycosylation of antibody Fc is described, e.g., in Routier, F. H., Glycoconjugate J. 14: 201-207 (1997). It will be appreciated that compositions comprising monoclonal antibodies are often heterogenous. As a practical matter, methods comprising administration of an afucosylated anti-FGFR2 antibody described herein may further comprise administering some antibody molecules that are not afucosylated. Within a population of antibodies, the antibodies are considered to be afucosylated if <5% of the antibodies of the population comprise fucose at Asn297. For example, in some embodiments, greater than 95% of the molecules of anti-FGFR2b antibody administered to the subject are afucosylated. For example, in some embodiments, at least 96%, 97%, or 99% of the molecules of anti-FGFR2b antibody administered to the subject may be afucosylated. Additional antibodies that may be used in embodiments herein include those described in US Patent Publication No. 2015/0050273, which describes certain afucosylated anti-FGFR2b antibodies, and which is incorporated herein by reference in its entirety.

In some embodiments, an afucosylated anti-FGFR2b antibody mediates antibody-dependent cell-mediated cytotoxicity (ADCC) in the presence of human effector cells more effectively than an antibody with the same amino acid sequence that comprises fucose. Generally, ADCC activity may be determined using the in vitro ADCC assay disclosed in U.S. Patent Publication No. 2015/0050273, but other assays or methods for determining ADCC activity, e.g. in an animal model etc., are contemplated.

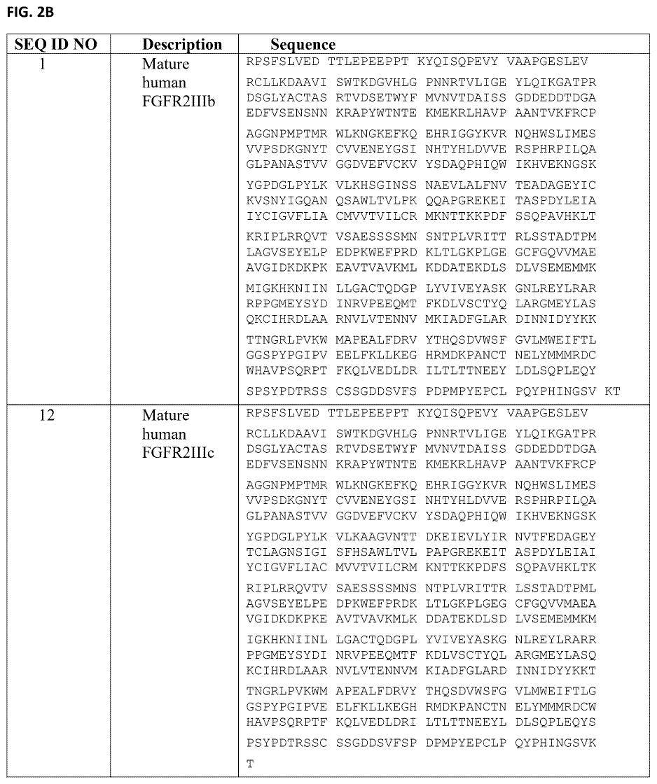

Example sequences of anti-FGFR2b antibodies of some embodiments are shown in FIG. 2A. In the method of some embodiments, the anti-FGFR2b antibody comprises at least one, two, three, four, five, or six complementarity determining regions (CDRs) selected from (a) a HCDR1 of SEQ ID NO: 6; (b) a HCDR2 of SEQ ID NO: 7; (c) a HCDR3 of SEQ ID NO: 8; (d) a LCDR1 of SEQ ID NO: 9; (e) a LCDR2 of SEQ ID NO: 10; and (f) a LCDR3 of SEQ ID NO: 11. The anti-FGFR2b may comprise a heavy chain comprising a heavy chain variable region comprising a HCDR1 of SEQ ID NO: 6, a HCDR2 of SEQ ID NO: 7, and a HCDR3 of SEQ ID NO: 8, and may further comprise a light chain comprising a light chain variable region comprising a LCDR1 of SEQ ID NO: 9, a LCDR2 of SEQ ID NO: 8, and a LCDR3 of SEQ ID NO: 9. In the method of some embodiments, the heavy chain variable region is at least 90% identical to SEQ ID NO: 4 and the light chain variable region is at least 90% identical to SEQ ID NO: 5. In the method of some embodiments, the heavy chain variable region is at least 95% identical to SEQ ID NO: 4 and the light chain variable region is at least 95% identical to SEQ ID NO: 5. In some embodiments, the heavy chain variable region comprises SEQ ID NO: 4 and the light chain variable region comprises SEQ ID NO: 5. In the method of some embodiments, the heavy chain comprises SEQ ID NO: 2 and the light chain comprises SEQ ID NO: 3. Any of the anti-FGFR2 antibodies described herein may be afucosylated. For example, the antibody may be an IgG1 or IgG3 antibody that lacks fucose at Asn297. In the method of some embodiments, the anti-FGFR2b antibody is bemarituzumab.

In the method of some embodiments, the anti-FGFR2b antibody comprises a heavy chain variable region comprising SEQ ID NO: 4 and a light chain variable region comprising SEQ ID NO: 5. It is further contemplated that in some embodiments, the anti-FGFR2b antibody comprises one or more substitutions, insertions, or deletions compared to SEQ ID NO: 4 and/or SEQ ID NO: 5, and continues to bind to FGFR2b. For example, the anti-FGFR2b antibody comprises one or more substitutions, insertions, or deletions compared to SEQ ID NO: 4 and/or SEQ ID NO: 5 and may bind to FGFR2b with an affinity, as measured by surface plasmon resonance, that is no less than an order of magnitude lower than the affinity of a reference anti-FGFR2b antibody comprising a heavy chain variable region comprising SEQ ID NO: 4 and a light chain variable region comprising SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 90% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 91% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 95% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region at least 97% identical to SEQ ID NO: 5. In the method of some embodiments, the anti-FGFR2b antibody comprises a heavy chain variable region at least 90%, 91%, 92%, 93%, 94%, 95%, 96%, 96%, 98%, or 99% identical to SEQ ID NO: 4 and a light chain variable region of SEQ ID NO: 5. In the method of some embodiments, the anti-FGFR2b antibody comprises a heavy chain variable region at least 90% identical to SEQ ID NO: 4 and a light chain variable region at least 90% identical to SEQ ID NO: 5. In the method of some embodiments, the heavy chain variable region is at least 95% identical to SEQ ID NO: 4 and the light chain variable region is at least 95% identical to SEQ ID NO: 5. In some embodiments, a total of 1 to 10 amino acids have been substituted, inserted and/or deleted in SEQ ID NO: 4. The substitutions, insertions, or deletions may occur in regions outside the CDRs (i.e., in the FRs). In the method of some embodiments, a total of 1 to 10, 1 to 5, or 1 to 3 amino acids have been substituted, inserted and/or deleted in SEQ ID NO: 5. The substitutions, insertions, or deletions may occur in regions outside the CDRs (i.e., in the FRs). In some embodiments, a total of 1 to 10, 1 to 5, or 1 to 3 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 10 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 5 amino acids have been substituted, inserted and/or deleted in SEQ ID NO: 4. The substitutions, insertions, or deletions may occur in regions outside the CDRs (i.e., in the FRs). In the method of some embodiments, up to 10, up to 5, or up to 3 amino acids have been substituted, inserted and/or deleted in SEQ ID NO: 5, and up to 3 amino acids have been substituted, inserted and/or deleted in SEQ ID NO: 4. The substitutions, insertions, or deletions may occur in regions outside the CDRs (i.e., in the FRs). In some embodiments, a total of 1 to 10, 1 to 5, or 1 to 3 amino acids have been substituted, inserted and/or deleted in SEQ ID NO: 4. Any of the anti-FGFR2 antibodies described herein may be afucosylated. For example, the antibody may be an IgG1 or IgG3 antibody that lacks fucose at Asn297.

Additional examples of anti-FGFR2b antibodies are the HuGAL-FR21, GAL-FR22 and GAL-FR23 antibodies described in U.S. Pat. No. 8,101,723 B2, incorporated by reference in its entirety herein. FIGS. 13 and 14 of U.S. Pat. No. 8,101,723 B2 show the amino acid sequences of the variable regions and full-length mature antibody chains of HuGAL-FR21, and are incorporated by reference herein. The heavy chain variable region sequences of antibody HuGAL-FR21, are underlined in FIG. 13 of U.S. Pat. No. 8,101,723 B2, and are specifically incorporated by reference herein. The light and heavy chain variable regions of GAL-FR22, for example, are provided as SEQ ID NOs: 7 and 8 in U.S. Pat. No. 8,101,723 B2, while the Kabat CDRs and the light and heavy chain variable regions are also provided in FIG. 16 of that patent, which are incorporated by reference herein. The GAL-FR21, GAL-FR22 and GAL-FR23 producing hybridomas are deposited at the American Type Culture Collection, PO Box 1549, Manassas VA, USA, 20108, as ATCC Numbers 9586, 9587, and 9408, on November 6, November 6, and Aug. 12, 2008, respectively. Thus, in some embodiments, the FGFR2 antibody is an antibody comprising the amino acid sequence of an antibody obtained from one of those three hybridoma strains.

Bemarituzumab is an afucosylated humanized monoclonal antibody that targets the fibroblast growth factor (FGF) receptor isoform 2b (FGFR2b) with a dual mechanism of FGF binding inhibition and antibody-dependent cellular cytotoxicity. The anti-FGFR2b antibody of any of the methods described herein may be bemarituzumab. Bemarituzumab comprises the heavy chain of SEQ ID NO: 2 and the light chain of SEQ ID NO: 3. In the method of some embodiments, the anti-FGFR2 antibody comprises the heavy chain of SEQ ID NO: 2 and the light chain of SEQ ID NO: 3, and is afucosylated. In the method of some embodiments, the anti-FGFR2b antibody is bemarituzumab. In the method of some embodiments, the anti-FGFR2b antibody comprises HCDR1-3 and LCDR1-3 of bemarituzumab. Bemarituzumab may be produced in a Chinese hamster ovary cell line that lacks the FUT8 gene, so that the produced antibody is glycosylated but lacks a core fucose in the polysaccharide portion of the antibody. The absence of the core fucose results in higher affinity for the Fc receptor FcγRIIIa compared to the fucosylated molecule and potentially enhances immune cell-mediated tumor cell killing.

Bemarituzumab inhibits FGF ligand-stimulated FGFR2b phosphorylation and cell proliferation in cell culture in FGFR2b overexpressing gastric and breast cancer cell lines. Bemarituzumab also inhibits tumor growth in FGFR2b overexpressing gastric and breast xenograft models. Without being limited by theory, it is contemplated that mechanisms of action of bemarituzumab may include blocking ligand binding and downstream signaling, decreasing expression of the FGFR2b driver protein, and/or enhancing ADCC. Furthermore, without being limited by theory, it is contemplated that since bemarituzumab is specific for the FGFR2b receptor, it does not interfere with signaling of the other FGFs/FGFRs, including FGFR2c. In contrast to the FGFR tyrosine kinase inhibitors (TKIs), bemarituzumab does not inhibit FGF23 signaling. FGF23 is a ligand involved in calcium/phosphate metabolism and therefore, treatment with bemarituzumab is not associated with the hyperphosphatemia associated with the FGFR TKIs (Catenacci et al, 2020; Dienstmann et al, 2014; Sequist et al, 2014; Andre et al, 2013; Brown et al, 2005).

Bemarituzumab monotherapy has been investigated in a phase 1 dose-finding study (FPA144-001) and in combination with mFOLFOX6 chemotherapy in FGFR2b-positive gastric cancer in the FIGHT study. Bemarituzumab efficacy correlated with the degree of FGFR2b overexpression by immunohistochemistry (IHC) in gastric cancer and has demonstrated a manageable safety profile in combination with mFOLFOX6. Genomic and IHC data suggest that other carcinomas including may also have a significant rate of FGFR2b overexpression.

Bemarituzumab blocks FGFR2b phosphorylation, downregulates the receptor, and inhibits downstream signaling. The effect on downstream signaling was measured by examining phosphorylation of a protein that is directly phosphorylated by the FGFR2 protein, FGFR substrate-2 (FRS2). Each of these mechanisms has been explored in vitro and in vivo and, without being limited by theory, appears to contribute to the anti-tumor activity of bemarituzumab. In FGFR2b-overexpressing human tumor xenograft models, bemarituzumab shows dose-related anti-tumor activity with regression and complete responses at well-tolerated doses.

Bemarituzumab demonstrated consistent pharmacokinetic (PK) behavior following intravenous (IV) administration in rats and cynomolgus monkeys, and the PK characteristics observed were consistent across all studies. The half-life was dose-dependent, ranging from 0.8 days at the lowest doses (1 to 1.5 mg/kg) to at least 8 days at the highest doses (100 to 150 mg/kg) tested in cynomolgus monkeys. Bemarituzumab demonstrated dose-dependent, nonlinear PK that was marked by a faster clearance at the terminal phase of the plasma concentration time profile and a greater than dose proportional increase in exposure (area under the concentration time curve [AUC]) with increasing dose. Target-mediated clearance was saturable, marked by dose-proportional increases in exposure at doses exceeding this level when dosed at weekly intervals. The PK studies supporting the toxicokinetic studies showed dose-dependent increases in exposure (AUCs) supporting the reliability of these studies to assess toxicity. Significant reproductive and developmental toxicities were observed at all dose levels (5 to 100 mg/kg/doses) in the embryo-fetal development with prenatal and postnatal development study. As such, it is contemplated that in some embodiments, subjects treated with bemarituzumab are not pregnant.

Bemarituzumab has demonstrated an acceptable safety profile. Identified risks when used in combination with mFOLFOX6 include corneal toxicity, infusion related reactions, gastrointestinal toxicity (stomatitis and mucosal inflammation), nail toxicity and increase in AST and ALT. Corneal events are very common with bemarituzumab with the most common adverse event being dry eye. Although nearly all of the events have been non-serious, grade 3 events such ulcerative keratitis and punctate keratitis which can lead to decreases in visual acuity have been observed. The majority of the corneal events typically resolve with treatment interruption or discontinuation and standard of care interventions for the corneal events. As such, it is contemplated that in some embodiments, subjects treated with bemarituzumab are further treated with ocular lubricants. The ocular lubricants may be administered prophylactically to reduce the risk of corneal events.

In some methods described herein, bemarituzumab may be provided in a drug product composition comprising or consisting essentially of an aqueous solution comprising 20 mg/mL bemarituzumab, L-histidine, sucrose, and polysorbate 20 at pH 6.0. For example, the solution may comprise or consist essentially of or consist of 20 mg/mL bemarituzumab, 20 mM L-histidine, 270 nM sucrose, and 0.01% (w/v) polysorbate 20 at pH 6.0

The anti-FGFR2b antibody, such as bemarituzumab, may be administered intravenously in methods described herein.

### CAPOX

CAPOX (also known as XELOX) is a chemotherapy regimen including capecitabine and oxaliplatin, which is indicated for the treatment of advanced stage colorectal cancer. The most common side effects for capecitabine (30%) include diarrhea, hand-and-foot syndrome, nausea, vomiting, abdominal pain, fatigue/weakness, and hyperbilirubinemia. Other adverse reactions, including serious adverse reactions, have been reported (capecitabine (XELODA®) Prescribing Information, 2021). The most common side effects for oxaliplatin (40%) include peripheral sensory neuropathy, neutropenia, thrombocytopenia, anemia, nausea, increase in transaminases and alkaline phosphatase, diarrhea, emesis, fatigue, and stomatitis.

Oxaliplatin may be administered intravenously in methods described herein, while capecitabine may be administered orally.

### SOX

SOX is a chemotherapy regimen including tegafur/gimeracil/oteracil (S-1) and oxaliplatin. S-1 combines tegafur, 5-chloro-2,4-dihydroxypyridine, and potassium oxonate. Tegafur is a metabolically activated prodrug of 5-FU, and 5-chloro-2,4-dihydropyridine (CDHP) and potassium oxonate (Oxo) are modulators: CDHP inhibits 5-FU degradation and Oxo suppresses activation of 5-FU in the gastro-intestinal (GI) tract by inhibiting its activation in mucosal cells (Shirasaka, 2008).

S-1 has been shown to be noninferior in efficacy to 5-FU monotherapy for previously untreated metastatic gastric cancer and SOX have been shown to be comparable in efficacy with an improved safety profile over S-1 and cisplatin (Boku et al, 2009; Yamada et al, 2014). The most common side effects of S-1 in combination with oxaliplatin include (≥40%) leukopenia, neutropenia, anemia, thrombocytopenia, AST and ALT elevation, diarrhea, nausea, fatigue, and sensory neuropathy.

Oxaliplatin may be administered intravenously in methods described herein, while S-1 may be administered orally.

### PD-1/PD-L1 Antibodies

In some embodiments, the disclosed methods further comprise administering an anti-PD-1 or anti PD-L1 antibody to the subject. The PD-1/PD-L1 axis is involved in the suppression of T cell immune responses in cancer. Antagonists of this pathway have been clinically validated across a number of solid tumor indications. Monoclonal antibodies that target the PD-1 pathway have been approved by the U.S. Food and Drug Administration (FDA) for the treatment of metastatic melanoma. Exemplary anti-PD-1 antibodies include, but are not limited to, nivolumab, pembrolizumab, and orcemiplimab. Exemplary anti-PD-L1 antibodies include, but are not limited to, atezolizumab, avelumab, and durvalumab.

Nivolumab, a PD-1 antibody that blocks interaction with PD-L1, has recently been approved in the U.S. in combination with fluoropyrimidine- and platinum-containing chemotherapy. Nivolumab (also known as “OPDIVO®”) is a fully human IgG4 (S228P) PD-1 immune checkpoint inhibitor antibody that selectively prevents interaction with PD-1 ligands (PD-L1 and PD-L2), thereby blocking the down-regulation of antitumor T-cell functions (U.S. Pat. No. 8,008,449; Wang et al., 2014 Cancer Immunol Res. 2(9):846-56).

### Methods of Treating Gastric Cancer

### Methods Comprising Administering an Anti-FGFR2b Antibody

Methods of treating gastric cancer, such as gastric adenocarcinoma or gastroesophageal junction adenocarcinoma, in a subject are described herein. The methods may comprise administering an anti-FGFR2b antibody to the subject Q3W at a dose of at least 15 mg/kg. The methods may further comprise administering an additional dose of the anti-FGFR2b antibody of 5-15 mg/kg, 7-10 days after a first administration of the Q3W administration of the anti-FGFR2b antibody. The additional dose may be referred to as an “intervening” dose. In some of the methods, the anti-FGFR2b antibody is administered to the subject Q3W at a dose of greater than 15 mg/kg to no more than 22 mg/kg (e.g., 15-22 mg/kg). In some of the methods, the anti-FGFR2b antibody is administered to the subject Q3W at a dose of about 15 mg/kg, about 17 mg/kg, about 20 mg/kg, about 22 mg/kg, about 25 mg/kg, or about 30 mg/kg, including ranges between any two of the listed values, for example, 15-17 mg/kg, 17-20 mg/kg, 17-22 mg/kg, 17-25 mg/kg, 20-22 mg/kg, 20-25 mg/kg, 22-25 mg/kg, 20-30 mg/kg, greater than 22 mg/kg to no more than 30 mg/kg, or greater than 22 mg/kg to no more than 25 mg/kg. In some of the methods, the anti-FGFR2b antibody is administered to the subject Q3W at a dose of 15 mg/kg. In accordance with some methods described herein, the additional dose or “intervening” dose may be 5-15 mg/kg (e.g., 10-15 mg/kg) and may be administered 7-10 days after the first administration of the Q3W administration of the anti-FGFR2b antibody. In accordance with some methods described herein, the additional dose or “intervening” dose may be 6-9 mg/kg, 7-8 mg/kg, 9-13 mg/kg, or 10-12 mg/kg and may be administered 7-10 days after the first administration of the Q3W administration of the anti-FGFR2b antibody. By way of example, the additional dose or “intervening” dose may be about 7 mg/kg, about 7.5 mg/kg, about 8 mg/kg, about 9 mg/kg, about 9.5 mg/kg, about 10 mg/kg, about 10.5 mg/kg, about 11 mg/kg, about 11.5 mg/kg, or about 12 mg/kg 7-10 days after the first administration of the Q3W administration of the anti-FGFR2b antibody. For any of the methods described herein, the anti-FGFR2b antibody may be bemarituzumab.

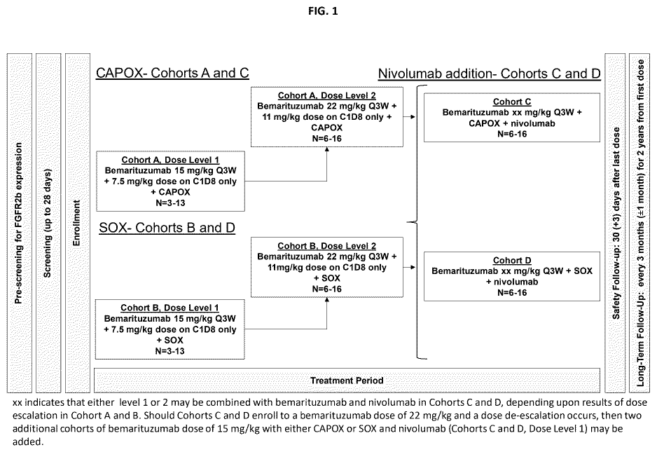

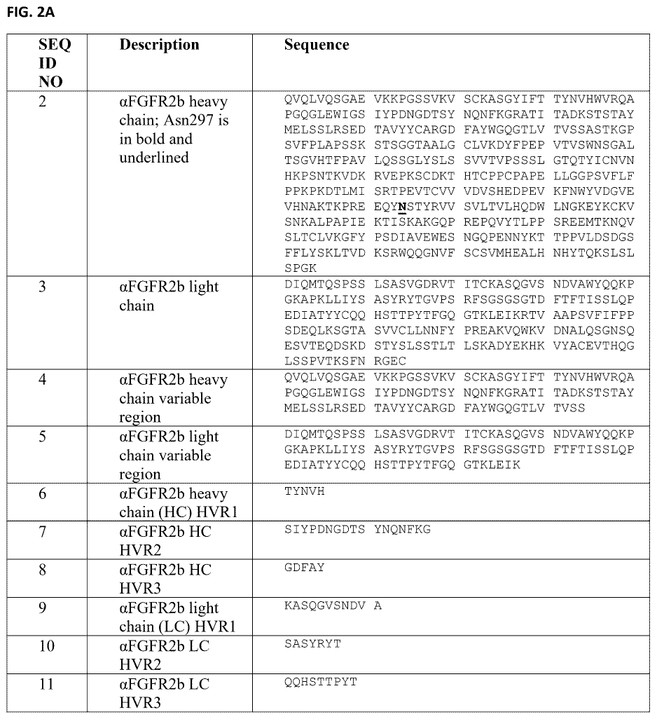

Methods of treating gastric cancer in a subject according to some embodiments are depicted in FIG. 1. The method may comprise administering an anti-FGFR2b antibody to the subject Q3W at a dose of at least 15 mg/kg or at least about 20 mg/kg, for example 15-22 mg/kg, 15-20 mg/kg, 15-17 mg/kg, 17-20 mg/kg, 17-22 mg/kg, 17-25 mg/kg, 20-22 mg/kg, 20-25 mg/kg, 22-25 mg/kg, 25-30 mg/kg, 20-30 mg/kg, about 15 mg/kg, about 17 mg/kg, about 20 mg/kg, about 22 mg/kg, about 25 mg/kg, or about 30 mg/kg. The method may further comprise, 7-10 days after the first administration of anti-FGFR2b antibody, administering to the subject an additional dose of the anti-FGFR2b antibody of 5-15 mg/kg or 10-15 mg/kg, for example 8 mg/kg, 7-7.5 mg/kg, 7.5-8 mg/kg, 7.5-9 mg/kg, or 7.5-10 mg/kg, 7-9 mg/kg, 7-10 mg/kg, 10-12 mg/kg, about 7 mg/kg, about 7.5 mg/kg, about 8 mg/kg, about 9 mg/kg, about 10 mg/kg, or about 11 mg/kg.

In some embodiments, the method may comprise administering an anti-FGFR2b antibody to the subject Q3W at a dose of about 15 mg/kg followed by administering an additional dose of the anti-FGFR2b antibody of about 7.5 mg/kg 7-10 days after the first administration of the anti-FGFR2b antibody. For example, the method may comprise administering an anti-FGFR2b antibody to the subject Q3W at a dose of about 15 mg/kg followed by administering an additional dose of the anti-FGFR2b antibody of about 7.5 mg/kg 8 days after the first administration of the anti-FGFR2b antibody. In other embodiments, the method may comprise administering an anti-FGFR2b antibody to the subject Q3W at a dose of about 22 mg/kg followed by administering an additional dose of the anti-FGFR2b antibody of about 11 mg/kg 7-10 days after the first administration of the anti-FGFR2b antibody. For example, the method may comprise administering an anti-FGFR2b antibody to the subject Q3W at a dose of about 22 mg/kg followed by administering an additional dose of the anti-FGFR2b antibody of about 11 mg/kg 8 days after the first administration of the anti-FGFR2b antibody.

### Methods Comprising Administering an Anti-FGFR2b Antibody and CAPOX (Oxaliplatin and Capecitabine) or SOX (Oxaliplatin and S-1)

In some embodiments, the methods described herein may further comprise administering oxaliplatin and capecitabine (CAPOX) to the subject. In other embodiments, the methods may further comprise administering oxaliplatin and S-1 (SOX). For any of the methods described herein, the anti-FGFR2b antibody may be bemarituzumab.

Methods of treating gastric cancer in a subject according to some embodiments are depicted in FIG. 1. The method may comprise administering a first administration of anti-FGFR2b antibody to the subject at a dose of at least 15 mg/kg, for example at least 20 mg/kg, at least 22 mg/kg, 25 mg/kg, 20-25 mg/kg, 20-30 mg/kg, or 25-30 mg/kg. The anti-FGFR2b antibody may be administered intravenously. The method may further comprise administering CAPOX to the subject Q3W, for example administering the oxaliplatin intravenously at a dose of at least 65 mg/m² Q3W (e.g., 65 mg/m²-150 mg/m² Q3W) and administering the capecitabine orally at a dose of at least 500 mg/m² (e.g., 500 mg/m²-1500 mg/m²) twice a day. By way of example, the oxaliplatin may be administered intravenously at a dose of about 100-150 mg/m² (e.g. 100-120 mg/m², 120-130 mg/m², 130-140 mg/m², or 140-150 mg/m²) Q3W, and the capecitabine may be administered orally at a dose of about 750-1500 mg/m² (e.g., 750-800 mg/m², 800-900 mg/m², 900-1000 mg/m², 1000-1100 mg/m², 1100-1200 mg/m², 1200-1300 mg/m², 1300-1400 mg/m², or 1400-1500 mg/m²) twice a day. Optionally, the method may further comprise, 7-10 days after the first administration of anti-FGFR2b antibody, administering to the subject an additional dose of the anti-FGFR2b antibody of 5-15 mg/kg or 10-15 mg/kg, for example, 8 mg/kg, 7-7.5 mg/kg, 7.5-8 mg/kg, 7.5-9 mg/kg, 7.5-10 mg/kg, 7-9 mg/kg, 7-10 mg/kg, 10-12 mg/kg, about 7 mg/kg, about 7.5 mg/kg, about 8 mg/kg, about 9 mg/kg, about 10 mg/kg, or about 11 mg/kg. For example, the method may comprise administering the anti-FGFR2b antibody intravenously Q3W at a dose of 15-22 mg/kg, administering an additional dose of the anti-FGFR2b antibody of 7-12 mg/kg 7-10 days after a first administration of the anti-FGFR2b antibody Q3W, administering oxaliplatin intravenously at a dose of at 100-150 mg/m², such as 130 mg/m² Q3W, and administering capecitabine orally at a dose of 750-1500 mg/m², such as 1000 mg/m², twice a day. In some embodiments, the method may comprise administering the anti-FGFR2b antibody intravenously Q3W at a dose of 22-30 mg/kg, and administering an additional dose of the anti-FGFR2b antibody of 10-12 mg/kg 7-10 days after a first administration of the anti-FGFR2b antibody Q3W, administering oxaliplatin intravenously at a dose of at 130 mg/m² Q3W and administering capecitabine orally at a dose of 1000 mg/m2 twice a day. Optionally, the oxaliplatin and anti-FGFR2b antibody may be administered Q3W at the same time, for example in a single composition, or in separate compositions. By way of example, the anti-FGFR2b antibody may be bemarituzumab.

In other embodiments, the method of treating gastric cancer may comprise administering a first administration of anti-FGFR2b antibody to the subject at a dose of at least 15 mg/kg, for example at least 20 mg/kg, at least 22 mg/kg, 25 mg/kg, 20-25 mg/kg, 20-30 mg/kg, or 25-30 mg/kg. The anti-FGFR2b antibody may be administered intravenously. The method may further comprise administering SOX to the subject Q3W, for example administering the oxaliplatin intravenously at a dose of at least 65 mg/m² Q3W (e.g., 65 mg/m²-150 mg/m² Q3W) and administering the S-1 orally at a dose of at least 20 mg/m² (e.g., 20 mg/m²−60 mg/m²) twice a day. By way of example, the oxaliplatin may be administered intravenously at a dose of about 100-150 mg/m² (e.g. 100-120 mg/m², 120-130 mg/m², 130-140 mg/m², or 140-150 mg/m²) Q3W, and the S-1 may be administered orally at a dose of about 30-60 mg/m² (e.g., 30 mg/m², 35 mg/m², 40 mg/m², 45 mg/m², 50 mg/m², 55 mg/m², 60 mg/m², or a range defined by any two of the foregoing values) twice a day. Optionally, the method may further comprise, 7-10 days after the first administration of anti-FGFR2b antibody, administering to the subject an additional dose of the anti-FGFR2b antibody of 5-15 mg/kg or 10-15 mg/kg, for example, 8 mg/kg, 7-7.5 mg/kg, 7.5-8 mg/kg, 7.5-9 mg/kg, 7.5-10 mg/kg, 7-9 mg/kg, 7-10 mg/kg, 10-12 mg/kg, about 7 mg/kg, about 7.5 mg/kg, about 8 mg/kg, about 9 mg/kg, about 10 mg/kg, or about 11 mg/kg. For example, the method may comprise administering the anti-FGFR2b antibody intravenously Q3W at a dose of 15-22 mg/kg, administering an additional dose of 7-12 mg/kg 7-10 days after a first administration of the anti-FGFR2b antibody Q3W, administering oxaliplatin intravenously at a dose of at 10-150 mg/m², such as 130 mg/m², Q3W, and administering S-1 orally at a dose of 30-60 mg/m², such as 40 mg/m², twice a day. In some embodiments, the method may comprise administering the anti-FGFR2b antibody intravenously Q3W at a dose of 22-30 mg/kg, administering an additional dose of 10-12 mg/kg 7-10 days after a first administration of the anti-FGFR2b antibody Q3W, administering oxaliplatin intravenously at a dose of at 130 mg/m² Q3W, and administering S-1 orally at a dose of 40 mg/m² twice a day. Optionally, the oxaliplatin and anti-FGFR2b antibody may be administered Q3W at the same time, for example in a single composition, or in separate compositions. By way of example, the anti-FGFR2b antibody may be bemarituzumab.

### Methods Comprising Administering Anti-FGFR2b Antibody, CAPOX or SOX, and an Anti-PD-1 or Anti-PD-L1 Antibody

As depicted in FIG. 1, the methods of treating gastric cancer describe herein may comprise administering an anti-FGFR2b antibody, CAPOX or SOX, and optionally an anti-PD-1 or anti PD-L1 antibody to the subject. The anti-PD-1 antibody or anti-PD-L1 antibody may be any such antibody known in the art or disclosed herein. In some embodiments, the method comprises administering an anti-PD-1 antibody. The anti-PD-1 antibody may be nivolumab. For example, the method of treating gastric cancer may comprise administering to the subject an anti-FGFR2b antibody, a chemotherapy regimen comprising: (a) oxaliplatin and capecitabine (CAPOX) or (b) oxaliplatin and S-1 (SOX), and nivolumab. In some embodiments, the method may comprise administering the anti-FGFR2b antibody Q3W at a dose of at least 15 mg/kg, administering an additional dose of the anti-FGFR2b antibody of 5-15 mg/kg 7-10 days after a first administration of the Q3W administration of the anti-FGFR2b antibody, administering oxaliplatin intravenously at a dose of at least 65 mg/m² Q3W, administering capecitabine orally at a dose of at least 500 mg/m² twice a day, and administering an anti-PD-1 antibody Q3W. In other embodiments, the method may comprise administering the anti-FGFR2b antibody Q3W at a dose of at least 20 mg/kg, administering an additional dose of the anti-FGFR2b antibody of 10-15 mg/kg 7-10 days after a first administration of the Q3W administration of the anti-FGFR2b antibody, administering oxaliplatin intravenously at a dose of at least 65 mg/m² Q3W, administering capecitabine orally at a dose of at least 500 mg/m² twice a day, and administering an anti-PD-1 antibody Q3W. In some embodiments, the method may comprise administering the anti-FGFR2b antibody Q3W at a dose of at least 15 mg/kg, administering an additional dose of the anti-FGFR2b antibody of 5-15 mg/kg 7-10 days after a first administration of the Q3W administration of the anti-FGFR2b antibody, administering oxaliplatin intravenously at a dose of at least 65 mg/m² Q3W, administering S-1 orally at a dose at a dose of at least 20 mg/m² twice a day, and administering an anti-PD-1 antibody Q3W. In other embodiments, the method may comprise administering the anti-FGFR2b antibody Q3W at a dose of at least 20 mg/kg, administering an additional dose of the anti-FGFR2b antibody of 10-15 mg/kg 7-10 days after a first administration of the Q3W administration of the anti-FGFR2b antibody, administering oxaliplatin intravenously at a dose of at least 65 mg/m² Q3W, administering S-1 orally at a dose at a dose of at least 20 mg/m² twice a day, and administering an anti-PD-1 antibody Q3W.

For any of the methods of treating gastric cancer described herein, the anti-PD-1 or anti-PD-L1 antibody may be administered intravenously on a Q3W regimen comprising any suitable dose. For example, an anti-PD-1 antibody, such as nivolumab, may be administered at a dose of about 240 mg-480 mg (e.g., 250 mg, 300 mg, 400 mg, or 450 mg). In some embodiments, the method comprises administering nivolumab at a dose of at least 300 mg Q3W, such as 300-400 mg Q3W (e.g., 310 mg, 320 mg, 330 mg, 340 mg, 350 mg, 360 mg, 370 mg, 380 mg, or 390 mg). For example, nivolumab may be administered at a dose of about 360 mg Q3W.

Described in accordance with methods of some embodiments is a study to evaluate the safety, tolerability, and efficacy of bemarituzumab in combination with CAPOX or SOX chemotherapy, optionally in further combination with an anti-PD-1 or anti PD-L1 antibody, in previously untreated advanced gastric cancer or gastroesophageal junction cancer. The study may comprise a dosing and scheduling of bemarituzumab and CAPOX or SOX, and optionally nivolumab, as described in the Examples.

For any of the methods treating gastric cancer described herein, cells of the gastric cancer of the subject may express FGFR2b. For example, cell of the gastric cancer of the subject may overexpress FGFR2b protein, overexpress FGFR2b mRNA, or comprise an FGFR2b gene amplification. In some methods, cells of the gastric cancer express FGFR2b protein as determined by immunohistochemistry (HC). For example, at least 5%, 10%, 15%, or 20% of the gastric cancer cells overexpress FGFR2b as determined by IHC. For example, the cells of the subject's gastric cancer may have an FGFR2b staining intensity of 2+ or 3+. For example, at least 5% (e.g., 5%, 10%, 15%, or 20%) of the gastric cancer cells may have an FGFR2b staining intensity of 1+, 2+ or 3+. If the cells of the subject's gastric cancer may have an FGFR2b staining intensity of 2+ or 3+, or if at least 5% of the gastric cancer cells may have an FGFR2b staining intensity of 1+, 2+ or 3+, the gastric cancer of the subject may be considered to overexpress FGFR2b. In some methods, the subject is determined to overexpress FGFR2b if any cells of the gastric cancer have an FGFR2b staining intensity of 2+ or 3+ as determined by IHC. It is contemplated that subjects having gastric cancer that overexpress FGFR2b are especially likely to benefit from methods of treatment comprising administering anti-FGFR2b antibodies (such as bemarituzumab) described herein. Optionally, cells of the gastric cancer are also assessed for PD-L1 expression, for example by IHC.

The following examples further illustrate the invention but, of course, should not be construed as in any way limiting its scope.

### Example 1: a Phase 1b Study of Bemarituzumab in Combination with Other Anti-Cancer Therapies in Previously Untreated Advanced Gastric or Gastroesophageal Junction Cancer with FGFR2b Overexpression

This example describes a phase 1b, open-label, multicenter study to evaluate the safety, tolerability, pharmacokinetics (PK), and efficacy of bemarituzumab in combination with other anti-cancer therapies in subjects with advanced gastric or gastroesophageal junction adenocarcinoma that express FGFR2b and who have not received prior treatment for unresectable or metastatic disease.

Tumor samples from subjects will be required to demonstrate FGFR2b overexpression prior to entering this study. Fibroblast growth factor receptor 2b (FGFR2b) overexpression will be centrally assessed by immunohistochemistry (IHC) on a tumor sample (either archival within 6 months of signing the pre-screening Informed Consent Form [ICF] or fresh biopsy).

The study includes a pre-screening period for FGFR2b testing, a 28-day screening period, a treatment period, a safety follow-up (SFU) visit, and a long-term follow-up (LTFU) period. Subjects will receive treatment until disease progression, unacceptable toxicity, subject request, or death (whichever comes first).

Subjects may continue nivolumab treatment beyond initial Response Evaluation Criteria in Solid Tumors (RECIST) v1.1 progressive disease (PD), as assessed by the investigator, as long as they meet all of the following criteria: (1) investigator assessed clinical benefit; (2) tolerance of study drug; and (3) stable Eastern Cooperative Oncology Group (ECOG) performance status.

Treatment beyond progression will not delay an imminent intervention to prevent serious complications of disease progression (i.e., central nervous system (CNS) metastases)

Subjects who discontinue all study treatments for any reason other than consent withdrawal, will undergo SFU visit approximately 28 (+3) days after the last dose of study treatment. In addition, subjects will undergo long term follow-up (LTFU) for survival approximately every 3 months (±1 month) for up to 2 years from the first dose of bemarituzumab.

Radiographic assessments are performed by the investigator according to RECIST v1.1 and will be performed every 6 weeks (±7 days) until week 56 and then every 12 weeks (±14 days). If a subject discontinues study treatment prior to radiographic disease progression, tumor assessments continue until radiographic progression or initiation of additional anticancer therapy. Primary and secondary objectives and endpoints are summarized in Table 1 below, and exploratory endpoints are summarized in Table 2.

TABLE 1

| | |
|---|---|
| Objectives | Endpoints |

Primary

| | |
|---|---|
| To evaluate the safety and | Dose-limiting toxicities (DLTs), |
| tolerability of | treatment-emergent adverse events, |
| bemarituzumab in combination | and clinically significant changes in |
| with other anti-cancer therapies | vital signs, physical examinations, |
| | electrocardiograms (ECGs), clinical |
| | laboratory tests, and visual acuity |

Secondary

| | |
|---|---|
| To characterize | Pharmacokinetic parameters for |
| the pharmacokinetics | bemarituzumab including, but not |
| (PK) of bemarituzumab in | limited to, area under the |
| combination with other | concentration-time curve (AUC), |
| anti-cancer therapies | maximum observed concentration |
| | (Cₘₐₓ), and observed concentration at |
| | the end of a dose interval (C_{trough}) |
| To evaluate preliminary | Objective response (defined as |
| anti-tumor activity | complete response [CR] + partial |
| of bemarituzumab in | response [PR]) as determined by |
| combination with other | investigator per Response Evaluation |
| anti-cancer therapies | Criteria in Solid Tumors version 1.1 |
| | (RECIST v1.1) |
| | Duration of response, defined as the |
| | time from first response to disease |
| | progression (as determined by |
| | investigator per RECIST v1.1) or |
| | death from any cause, whichever |
| | comes first |
| | Disease control defined as CR + PR + |
| | stable disease (SD) |
| | Progression-free survival (PFS), |
| | defined as time from enrollment until |
| | disease progression or death from any |
| | cause. Subjects alive without |
| | progression will be censored at their |
| | last evaluable disease assessment. |
| | Progression will be based on |
| | assessment by investigator per |
| | RECIST v1.1 |
| | Overall survival (OS), defined as time |
| | from enrollment until death from any |
| | cause. Subjects still alive will be |
| | censored at the date last known to be |
| | alive |

TABLE 2
Exploratory

| | |
|---|---|
| To evaluate the expression of | Expression of biomarkers, |
| biomarkers in blood and tumor | including, but not limited to, |
| samples | programmed death -receptor 1 |
| | (PD-L1) and Fibroblast growth |
| | factor receptor 2b (FGFR2b) |
| To evaluate the relationship | Correlation of biomarkers, |
| between exploratory biomarkers | including PD-L1 and FGFR2b |
| and efficacy | expression with efficacy endpoints |
| To evaluate the immunogenicity | Anti-bemarituzumab antibody formation |
| of bemarituzumab | |

The study explores the dose of bemarituzumab in combination with other anti-cancer therapies. The following combinations are being evaluated:
Cohort A: Bemarituzumab with CAPOX
Cohort B: Bemarituzumab with SOX
Cohort C: Bemarituzumab with CAPOX and nivolumab
Cohort D: Bemarituzumab with SOX and nivolumab.

Subjects will be enrolled in groups of 3 to 6 per dose level per cohort, and escalation will be guided primarily by safety responses to different doses. Cohorts B and D will not enroll subjects in the United States (US) and are outside the scope of the US Food and Drug Administration Investigational New Drug application. Cohorts A and B may start simultaneously.

The dose levels for bemarituzumab are:
Dose Level 1: bemarituzumab 15 mg/kg intravenously (IV) every 3 weeks (Q3W) with 7.5 mg/kg on cycle 1 day 8, 21-day cycles; and
Dose Level 2: bemarituzumab 22 mg/kg IV Q3W with 11 mg/kg on cycle 1 day 8, 21-day cycles.

Dose Level 2 within cohort A or B will be initiated once the dose level review team (DLRT) has deemed Dose Level 1 in that same combination cohort safe and tolerable.

Once the DLRT has deemed a Dose Level in Cohort A or B safe and tolerable, enrollment will commence in Cohort C or D, to evaluate the safety and tolerability of the combination studied in the Cohort A or B in combination with nivolumab.

For Cohort C and D, if dose level 2 of bemarituzumab is evaluated as a starting dose, Dose Level 1 may be used as de-escalation dose level to be enrolled if criteria are met for dose de-escalation.

The Bayesian Optimal Interval (BOIN) guideline with a target toxicity rate of 0.25 is used to determine escalation/de-escalation for each cohort. Alternative dose levels or dosing schedule(s) of bemarituzumab may be explored based on emerging safety, and PK data.

### Number of Subjects

Nine subjects each will be enrolled in Cohorts A and B (3 subjects at dose level 1 and then 6 subjects at dose level 2) and 6 subjects each in Cohorts C and D at the dose level selected from Cohorts A and B. Up to 10 additional subjects may be enrolled in a given dose level for a given cohort to further evaluate safety. Therefore, the minimum number of subjects studied is 30. Should a dose de-escalation occur after the first 3 subjects have been enrolled in cohorts C or D, then 9 subjects in each of these cohorts will be enrolled initially rather than 6 (3 at the dose level selected from Cohorts A and B and 6 at the dose level below this), bringing the study maximum to 96 subjects. The study may be amended at a later date to include additional combination cohorts and/or subjects for further safety or efficacy evaluation.

### Summary of Subject Eligibility Criteria

Subjects are (≥18 years) of age with histologically documented unresectable, locally advanced or metastatic gastric or gastroesophageal junction adenocarcinoma (not amenable to curative therapy) with FGFR2b overexpression. Subjects must be a candidate to receive the combination therapy for a given cohort (e.g., CAPOX or SOX with or without nivolumab), have evaluable disease (measurable or non-measurable) per RECIST v1.1, and must not have received prior treatment for unresectable or metastatic disease. Subjects with positive HER2 status will be excluded.

Once consented to the study, subjects will provide a medical history and undergo screening safety tests to confirm all eligibility requirements of the study have been met.

### Treatments

Bemarituzumab is administered as an approximately 30 minute (±10 minutes) IV infusion via peripheral vein or central venous catheter (21-day cycles). For the first dose level, 15 mg/kg bemarituzumab is administered IV every 21 days on day 1 (D1) of each cycle with a single dose of 7.5 mg/kg administered on cycle 1 Day 8. For the second dose level, 22 mg/kg bemarituzumab is administered IV every 21 days on D1 of each cycle with a single dose of 11 mg/kg administered on cycle 1 Day 8.

For CAPOX (21-day cycles), oxaliplatin administration will commence on day 1 of each cycle, after the end of the bemarituzumab (cohort A) or nivolumab (cohort C) infusion. On day 1, 130 mg/m² oxaliplatin is administered IV over 120 minutes. On day 1 through day 14, 1000 mg/m² capecitabine is administered orally twice daily (i.e., 1000 mg/m² in the morning and 1000 mg/m² in the evening).

For SOX (21-day cycles), oxaliplatin administration will commence on day 1 of each cycle, after the end of the bemarituzumab (cohort B) or nivolumab (cohort D) infusion. On day 1, 130 mg/m² oxaliplatin is administered IV over 120 minutes. On day 1 through day 14, 40 mg/m² S-1 is administered orally twice daily (i.e., 40 mg/m² in the morning and 40 mg/m² in the evening).

Nivolumab administration (21-day cycles) will commence on D1 of each cycle, after the end of bemarituzumab infusion. On day 1, 360 mg Nivolumab is administered IV over 30 minutes (±10 minutes).

### Rationale for Bemarituzumab Dose

The proposed dose level 1 and dose level 2 in this study are 15 mg/kg IV Q3W with 1 additional dose of 7.5 mg/kg on cycle 1 day 8, and 22 mg/kg IV Q3W with 1 additional dose of 11 mg/kg on cycle 1 day 8, respectively. Every 3-week dosing (Q3W) will be used in this study instead of every 2 week (Q2W) dosing to align with the CAPOX and SOX Q3W regimens. The selection of the target Q2W dose which was used to support the selection of the Q3W dose in this study is summarized below. The proposed starting dose (dose level 1) in this study is the target Q2W dose of bemarituzumab (15 mg/kg Q2W with 1 additional dose of 7.5 mg/kg on Cycle 1 Day 8) evaluated in FPA144-004, which will continue to be evaluated in phase 3 studies (20210096, 20210098), however with a decreased dosing frequency of Q2W to Q3W. Dose level 2 was chosen as a proportional increase of the target Q2W dose to account for the increased Q3W dosing interval in the linear dose range. Dose level 2 is projected to match the observed concentration at the end of a dose interval at steady state (C_{trough,ss}) and the area under the serum concentration versus time curve during the dosing interval at steady state divided by the dosing interval (C_{avg,ss}) of the target Q2W dose, and is expected to be efficacious if safety allows when maximum observed concentration (Cₘₐₓ) is higher than the target Q2W dose. In summary, the dose selected for the dose level 2 is primed to balance safety and efficacy based on observed safety and efficacy data from FPA144-004 study.

The FPA144-004 study with Q2W dosing of bemarituzumab at 15 mg/kg with 1 additional dose of 7.5 mg/kg on cycle 1 day 8 in combination with mFOLFOX6 achieved pre-specified statistical significance across all three of its prespecified efficacy endpoints including ORR, PFS, and OS in subjects with FGFR2b-positive, non-HER2 positive previously untreated advanced gastric or gastroesophageal junction cancer. Population PK analysis using PK data from studies FPA144-001, FPA144-002, and FPA144-004 indicated that no covariate was expected to have clinically meaningful effects on bemarituzumab exposure although baseline body weight, baseline albumin, gender, and combination therapy were identified as statistically significant covariates on the PK of bemarituzumab. No dose adjustment based on any of these factors is warranted in this population in combination with mFOLFOX6. Exposure-response analysis indicated 15 mg/kg Q2W with 1 additional dose of 7.5 mg/kg on cycle 1 day 8 adequately balanced the safety and efficacy. Therefore, 15 mg/kg every 2 weeks with 1 additional dose of 7.5 mg/kg on cycle 1 day 8 was selected to continue to be tested in combination with mFOLFOX6 in phase 3 studies as the target Q2W dose.

### Rationale for Capecitabine and Oxaliplatin (CAPOX) Dose

Capecitabine will be administered at 1000 mg/m² orally twice a day (i.e., 1000 mg/m² in the morning and 1000 mg/m² in the evening) for 14 days followed by 1 week of rest. Capecitabine should be taken with food. Oxaliplatin will be administered at 130 mg/m² IV on day 1 of each cycle. This regimen is a standard of care and has been studied in patients with advanced gastric and/or gastroesophageal junction cancer (Park et al, 2008; Kim et al, 2012; National Comprehensive Cancer Network, 2021).

### Rationale for Oxaliplatin and S-1 (SOX) Dose

S-1 will be administered at 40 mg/m² orally twice a day (i.e., 40 mg/m² in the morning and 40 mg/m² in the evening) or per dose banding adjustment per body surface area (BSA) per local standard of care for 14 days followed by 1 week of rest. Oxaliplatin will be administered at 130 mg/m² IV on day 1 of each cycle. This regimen is a standard of care in Japan and has been studied in patients with advanced gastric and/or gastroesophageal junction cancer (Boku et al, 2019; Kim et al, 2012).

### Rationale for Nivolumab Dose

Nivolumab will be administered at 360 mg IV Q3W. This is the approved dose for nivolumab in combination with fluoropyrimidine and platinum containing chemotherapy for subjects with metastatic gastric cancer, gastroesophageal junction cancer, and esophageal adenocarcinoma.

### Statistical Considerations

### Sample Size Considerations

The probability of observing at least 1 DLT if the true DLT rate is 30% with 3 subjects treated at dose level in a given cohort is a 66%; with 6 subjects the probability increases to 89%; with 13 or more subjects the probability is over 99%.

### Interim DLRT Reviews

For each cohort, the DLRT will convene to review all available safety, tolerability, laboratory, and PK data after subjects in groups of 3 to 6 have completed the 21-day DLT evaluation period during dose exploration and 21 days following the last subject enrolled in a dose cohort.

### Primary and Final Analyses

The primary analysis is planned after the last subject has completed the SFU period. A final analysis is planned after all subjects have ended the study.

### Analytical Methods

Descriptive statistics are provided for selected demographics, safety, PK, efficacy, and biomarker data. Descriptive statistics on continuous data include means, medians, standard deviations and ranges, while categorical data are summarized using frequency counts and percentages. Response rates are presented with 95% exact CI. Time-to-event endpoints are summarized using the Kaplan-Meier method.

### End of Study

An individual subject is considered to have completed the study if he/she has completed the last visit as shown in the schedule of Table 3. The end of study date is defined as the date when the last subject across all sites is assessed or receives an intervention for evaluation in the study (i.e., last subject last visit), including any additional parts in the study (e.g., LTFU, antibody testing), as applicable.

TABLE 3
Schedule of Activities

| | | | | | | Cycle 3 and Subsequent Cycles | | | | | | | |
|---|---|---|---|---|---|---|---|---|---|---|---|---|---|
| | | SCR | Cycles | | | | Q2C (odd | Q6W | As | | | | |
| Procedure^{a} | Pre- | −28 | 1 and 2 | | | Q1C | subsequent | after | clinically | Bema | | | |
| DAY | SCR | to 0 | C1D1 | C1D8 | C2D1 | D 1 | cycles) D 1 | C1D1 | indicated | EoT^{b} | SFU^{c} | LTFU^{d} | Notes |
| Pre-screening | X | | | | | | | | | | | | Pre-screening informed consent |
| informed consent | | | | | | | | | | | | | must be obtained prior to |
| | | | | | | | | | | | | | provision of tumor sample. |
| Archival tissue for IHC | X | | | | | | | | | | | | Provision of archival tissue (or |
| FGFR2b | | | | | | | | | | | | | fresh biopsy if archival tissue is |
| | | | | | | | | | | | | | not available) for assessment of |
| | | | | | | | | | | | | | FGFR2b overexpression testing |
| | | | | | | | | | | | | | by IHC is required. Fibroblast |
| | | | | | | | | | | | | | growth factor receptor 2b |
| | | | | | | | | | | | | | overexpression status by central |
| | | | | | | | | | | | | | IHC testing is required for |
| | | | | | | | | | | | | | enrollment. If archival, sample |
| | | | | | | | | | | | | | must be up to 6 months old as of |
| | | | | | | | | | | | | | the day of signing pre-screening |
| | | | | | | | | | | | | | consent form. |
| PD-L1 testing | X | | | | | | | | | | | | Utilizes leftover archival tissue |
| | | | | | | | | | | | | | (or fresh biopsy if archival tissue |
| | | | | | | | | | | | | | not available) from assessment |
| | | | | | | | | | | | | | of FGFR2b overexpression, if |
| | | | | | | | | | | | | | available. Programmed death- |
| | | | | | | | | | | | | | receptor 1 (PD-L1) will only be |
| | | | | | | | | | | | | | tested if FGFR2b |
| | | | | | | | | | | | | | overexpression is confirmed. |
| Informed consent | | X | | | | | | | | | | | Signed after FGFR2b positive |
| | | | | | | | | | | | | | status is confirmed |
| Eligibility criteria | | X | | | | | | | | | | | |
| Medical history | | X | | | | | | | | | | | Includes medical/surgical and |
| | | | | | | | | | | | | | cancer history (including local |
| | | | | | | | | | | | | | tumor biomarker testing results, |
| | | | | | | | | | | | | | if completed), ophthalmologic |
| | | | | | | | | | | | | | history, and prior medications |
| | | | | | | | | | | | | | that were being taken/used from |
| | | | | | | | | | | | | | 3 months prior to enrollment |
| Demographics | X | | | | | | | | | | | | |
| Physical exam | | X | X | X | X | (X) | | | | | X | | Prior to study drug |
| | | | | | | | | | | | | | administration in all cycles |
| | | | | | | | | | | | | | (X): Must include weight and |
| | | | | | | | | | | | | | body surface area (BSA) on day |
| | | | | | | | | | | | | | 1 of every cycle. |
| Height | | X | | | | | | | | | | | |
| Weight | | X | X | X | X | X | | | | | | | |
| ECOG PS | | X | | | | | X | | | | X | | Day 1 of every odd cycle |
| Vital status | | | | | | | | | | | | X | Every 3 months (±1 month) |
| | | | | | | | | | | | | | after SFU visit |
| Concomitant | | X | ------------------------------------X-------------------------------------- | | | | | | | | | (X) | (X): Collect information on |
| medications | | | | | | | | | | | | | ophthalmologic medication only |
| Subsequent | | | | | | | | | | | | X | Record subsequent therapies |
| anticancer therapy | | | | | | | | | | | | | taken for gastric or |
| | | | | | | | | | | | | | gastroesophageal junction |
| | | | | | | | | | | | | | cancer |
| Vital signs | | X | X | X | X | X | | | | | X | | Prior to study drug |
| | | | | | | | | | | | | | administration in all cycles |
| 12-lead ECG | | X | | | | | | | X | | | | 12-lead ECG performed after |
| | | | | | | | | | | | | | subject rests for 5 minutes prior |
| | | | | | | | | | | | | | to recording |
| Ophthalmology exam | | X | | | | | | (X) | X | | X | | Includes best distance corrected |
| | | | | | | | | | | | | | visual acuity, (Snellen or |
| | | | | | | | | | | | | | equivalent), ocular surface |
| | | | | | | | | | | | | | staining, slit lamp examination of |
| | | | | | | | | | | | | | anterior segment, and dilated |
| | | | | | | | | | | | | | retinal examinations or 3 field |
| | | | | | | | | | | | | | retinal photographs. |
| | | | | | | | | | | | | | Examinations may occur up to 5 |
| | | | | | | | | | | | | | days before dosing. |
| | | | | | | | | | | | | | (X) As above except dilated |
| | | | | | | | | | | | | | retinal examination or 3 field |
| | | | | | | | | | | | | | photographs are to be |
| | | | | | | | | | | | | | performed at every other |
| | | | | | | | | | | | | | ophthalmology exam. All |
| | | | | | | | | | | | | | examinations are to be |
| | | | | | | | | | | | | | performed every 6 weeks |
| | | | | | | | | | | | | | (−5 days) for the first 24 weeks, |
| | | | | | | | | | | | | | and then every 8 weeks (−5 |
| | | | | | | | | | | | | | days) thereafter. |
| Adverse events | | | ------------------------------------X-------------------------------------- | | | | | | | | | | Ocular symptoms should be |
| | | | | | | | | | | | | | reported as adverse events |
| | | | | | | | | | | | | | regardless of causality |
| Serious adverse | (X) | --------------------------------------------------------X------------------------------------------ | | | | | | | | | | [X] | (X): In pre-screening, only SAEs |
| events | | ---------- | | | | | | | | | | | related to fresh biopsy |
| | | | | | | | | | | | | | [X]: During LTFU, only serious |
| | | | | | | | | | | | | | adverse events suspected to be |
| | | | | | | | | | | | | | related to investigational product |
| | | | | | | | | | | | | | or any fatal adverse event |
| | | | | | | | | | | | | | (regardless of causality) that |
| | | | | | | | | | | | | | the investigator becomes aware |
| | | | | | | | | | | | | | of will be reported within |
| | | | | | | | | | | | | | 24 hours following the |
| | | | | | | | | | | | | | investigator's awareness of |
| | | | | | | | | | | | | | the event on the Event eCRF. |
| Adverse events of | | X | ---------------------------------------------------------X-------------------------------- | | | | | | | | | X | Ophthalmological events of any |
| special interest | | ------------------------ | | | | | | | | | | | grade occurring up to 100 days |
| | | | | | | | | | | | | | after SFU visit should be |
| | | | | | | | | | | | | | reported as adverse events to |
| | | | | | | | | | | | | | the Sponsor. Any |
| | | | | | | | | | | | | | ophthalmological events not |
| | | | | | | | | | | | | | resolved at the SFU visit must |
| | | | | | | | | | | | | | be followed until resolution or no |
| | | | | | | | | | | | | | further improvement is expected |
| | | | | | | | | | | | | | based on an ophthalmology |
| | | | | | | | | | | | | | assessment. |
| Radiological/tumor | | X | | | | | | (X) | | | [X] | ([X]) | (X): Every 6 (±7 days) week |
| assessments | | | | | | | | | | | | | from cycle 1 day 1 until week 56, |
| | | | | | | | | | | | | | then every 12 (±14 days) |
| | | | | | | | | | | | | | weeks. |
| | | | | | | | | | | | | | [X]: Can be omitted if last scan |
| | | | | | | | | | | | | | was performed <6 weeks |
| | | | | | | | | | | | | | (−3 days) prior to SFU visit or if |
| | | | | | | | | | | | | | tumor progression was |
| | | | | | | | | | | | | | previously determined. |
| | | | | | | | | | | | | | ([X]): Imaging assessments will |
| | | | | | | | | | | | | | continue for subjects who |
| | | | | | | | | | | | | | discontinue study treatment |
| | | | | | | | | | | | | | without disease progression. |
| Hematology | | X | X | X | X | X | | | X | | X | | Local test results must be |
| Chemistry panel | | X | X | X | X | X | | | X | | X | | obtained up to 72 hours prior to |
| | | | | | | | | | | | | | dose on dosing days for both |
| | | | | | | | | | | | | | bemarituzumab and |
| | | | | | | | | | | | | | chemotherapy. Lab results must |
| | | | | | | | | | | | | | be reviewed prior to study drug |
| | | | | | | | | | | | | | administration. |
| Thyroid Function | | X | | | | | X | | X | | | X | Applicable to Cohort C, D. |
| Testing | | | | | | | | | | | | | |
| Coagulation | | X | X | | X | | | | X | | | | |
| Urinalysis | | | X | | X | | | | X | | | | |
| Pregnancy test | | X | X | | | X | | | X | | X | | Highly sensitive serum β-hCG |
| (FCBP only) | | | | | | | | | | | | | results are required at screening |
| | | | | | | | | | | | | | and within 72 hours prior to |
| | | | | | | | | | | | | | cycle 1 day 1. Repeat |
| | | | | | | | | | | | | | pregnancy tests (urine is |
| | | | | | | | | | | | | | acceptable) are required day 1 |
| | | | | | | | | | | | | | of every cycle. |
| Amylase/lipase | | X | | | | | | | X | | | | |
| Immunogenicity | | | X | | X | | (X) | | | X | | | Anti-bemarituzumab blood |
| sampling | | | | | | | | | | | | | samples will be collected ≤4 |
| | | | | | | | | | | | | | hours prior to dosing of |
| | | | | | | | | | | | | | bemarituzumab. |
| | | | | | | | | | | | | | (X) To be collected for cycles 5 |
| | | | | | | | | | | | | | and 9. |
| PK samples (pre- | | | X | X | X | | (X) | | | | | | Pharmacokinetic samples will be |
| dose) | | | | | | | | | | | | | collected ≤4 hours prior to |
| | | | | | | | | | | | | | dosing of bemarituzumab. |
| | | | | | | | | | | | | | (X): Additional PK samples will |
| | | | | | | | | | | | | | be collected ≤4 hours prior to |
| | | | | | | | | | | | | | dosing of bemarituzumab for |
| | | | | | | | | | | | | | cycles 3 (504 hours collection for |
| | | | | | | | | | | | | | cycles 1 and 2 are the same as |
| | | | | | | | | | | | | | predose cycles 2 and 3, |
| | | | | | | | | | | | | | respectively), 5, and 9. |
| PK samples (post- | | | X | X | X | | (X) | | | [X] | | | Pharmacokinetic samples will be |
| dose) | | | | | | | | | | | | | collected at the following times |
| | | | | | | | | | | | | | after end of infusion of |
| | | | | | | | | | | | | | bemarituzumab: 15 (+10) min, 3 |
| | | | | | | | | | | | | | hours (±30 min), 6 hours (±1 |
| | | | | | | | | | | | | | hour), 24 hours (Day 2, ±1 |
| | | | | | | | | | | | | | hour), 72 hours (Day 4, ±6 |
| | | | | | | | | | | | | | hours), 168 hours (15 [+10] |
| | | | | | | | | | | | | | minutes at end of infusion on |
| | | | | | | | | | | | | | Cycle 1 Day 8; ±24 hours on |
| | | | | | | | | | | | | | Cycle 2 Day 8), 336 hours (Day |
| | | | | | | | | | | | | | 15, ±24 hours) and 504 hours |
| | | | | | | | | | | | | | (Day 22, ±24 hours) for cycles 1 |
| | | | | | | | | | | | | | and 2. |
| | | | | | | | | | | | | | (X): Additional PK samples will |
| | | | | | | | | | | | | | be collected 15 (+10) min after |
| | | | | | | | | | | | | | end of infusion for cycles 3, 5, |
| | | | | | | | | | | | | | and 9. |
| | | | | | | | | | | | | | [X]: Final sample will be |
| | | | | | | | | | | | | | collected 28 (+3) days after last |
| | | | | | | | | | | | | | dose of bemarituzumab (may be |
| | | | | | | | | | | | | | collected at SFU if all treatment |
| | | | | | | | | | | | | | is discontinued at same time). |
| Plasma ctDNA | | | (X) | | (X) | | (X) | | | X | | | (X) Pre-dose C1D1, C2D1, |
| Biomarker | | | | | | | | | | | | | C3D1 only. |
| | | | | | | | | | | | | | Cell pellet from plasma also |
| | | | | | | | | | | | | | collected. |
| Archived tumor tissue | | (X) | | | [X] | | | | | | [X] | | (X) Subjects to provide archived |
| (FFPE) or tumor | | | | | | | | | | | | | FFPE samples (collected within |
| biopsy (optional, as | | | | | | | | | | | | | 5 years, or leftover material from |
| applicable) | | | | | | | | | | | | | pre-screening) or undergo tumor |
| | | | | | | | | | | | | | biopsy for optional exploratory |
| | | | | | | | | | | | | | analysis. |
| | | | | | | | | | | | | | [X] Optional tumor biopsy for |
| | | | | | | | | | | | | | exploratory biomarker testing will |
| | | | | | | | | | | | | | be performed for subjects who |
| | | | | | | | | | | | | | consent to this assessment. It |
| | | | | | | | | | | | | | will be performed, if feasible, at |
| | | | | | | | | | | | | | screening, on cycle 2 day 1 and |
| | | | | | | | | | | | | | at the SFU or at time of |
| | | | | | | | | | | | | | progression. |
| Bemarituzumab | | | (X) | (X) | (X) | [X] | | | | | | | Bemarituzumab is to be |
| administration | | | | | | | | | | | | | administered Q3W, prior to all |
| | | | | | | | | | | | | | other study treatments. After |
| | | | | | | | | | | | | | cycle 1, bemarituzumab dose |
| | | | | | | | | | | | | | should be recalculated only if the |
| | | | | | | | | | | | | | weight changes >10% from |
| | | | | | | | | | | | | | cycle 1 day 1. If the dose is |
| | | | | | | | | | | | | | recalculated, the weight used for |
| | | | | | | | | | | | | | the recalculated dose should |
| | | | | | | | | | | | | | function as the new baseline. |
| | | | | | | | | | | | | | Cycle 1 day 1 must occur within |
| | | | | | | | | | | | | | 3 days of enrollment. |
| | | | | | | | | | | | | | (X): For cycles 1 through 3, day |
| | | | | | | | | | | | | | 1 dose should be administered |
| | | | | | | | | | | | | | every 21 days, regardless of |
| | | | | | | | | | | | | | delays in other anti-cancer |
| | | | | | | | | | | | | | treatment. |
| | | | | | | | | | | | | | [X]: Starting at cycle 4, may be |
| | | | | | | | | | | | | | delayed up to 7 days to be |
| | | | | | | | | | | | | | synchronized with other anti- |
| | | | | | | | | | | | | | cancer treatment. |
| Capecitabine | | | X | | X | X | | | | | | | Cohorts A and C only: Day 1 |
| administration | | | | | | | | | | | | | through 14 of each cycle |
| | | | | | | | | | | | | | (evening of day 1 to morning of |
| | | | | | | | | | | | | | day 15) |
| S-1 Administration | | | X | | X | X | | | | | | | Cohorts B and D only: Day 1 |
| | | | | | | | | | | | | | through 14 of each cycle |
| | | | | | | | | | | | | | (evening of day 1 to morning of |
| | | | | | | | | | | | | | day 15) |
| Oxaliplatin | | | X | | X | X | | | | | | | Q3W, Cohort A and B: |
| administration | | | | | | | | | | | | | Administered after |
| | | | | | | | | | | | | | bemarituzumab |
| | | | | | | | | | | | | | Cohort C and D: Administered |
| | | | | | | | | | | | | | after nivolumab |
| | | | | | | | | | | | | | If CAPOX or SOX are delayed, |
| | | | | | | | | | | | | | vital signs and clinical laboratory |
| | | | | | | | | | | | | | tests must be performed within |
| | | | | | | | | | | | | | 72 hours prior to administration. |
| Nivolumab | | | X | | X | X | | | | | | | Cohort C and D only: |
| administration | | | | | | | | | | | | | Administered after |
| | | | | | | | | | | | | | bemarituzumab and prior to |
| | | | | | | | | | | | | | oxaliplatin. |

### Study Population

Investigators will maintain a screening log of all potential study candidates that includes limited information about the potential candidate (e.g., date of screening). This log may be completed and updated via an Interactive Response Technology (TRT). Eligibility criteria is evaluated during screening. Before any study-specific activities/procedures, the appropriate written informed consent must be obtained. Prospective approval of protocol deviations to recruitment and enrollment criteria, also known as protocol waivers or exemptions will not be provided.

### Inclusion Criteria

Subjects are eligible to be included in the study only if all of the following criteria apply:
Subject has provided informed consent prior to initiation of any study specific activities/procedures.
Age ≥18 years (or legal adult age within country, whichever is older) at the time that the ICF is signed.
Pathologically confirmed gastric or gastroesophageal junction adenocarcinoma.
Disease that is unresectable, locally advanced, or metastatic (not amenable to curative therapy).
Tumor overexpresses FGFR2b as determined by centrally performed IHC testing.
Eastern Cooperative Oncology Group Performance Score (ECOG-PS) 0 to 1.
Adequate organ function, defined as follows:
   Absolute neutrophil count ≥1.5×10⁹/L
   Platelet count ≥100×10⁹/L
   Hemoglobin 9 g/dL
AST and ALT <3× upper limit of Normal [ULN] (or <5×ULN if liver involvement). Total bilirubin <1.5×ULN (or <2×ULN if liver involvement; with the exception of subjects with Gilbert's disease)
Calculated or measured creatinine clearance (CrCl) of ≥50 mL/minute calculated using the formula of Cockcroft and Gault ([140−Age]×Mass [kg]/[72×Creatinine mg/dL]) (×0.85 if female). Twenty-four hour urine collection is not required but is allowed.
International normalized ratio (INR) or prothrombin time (PT)<1.5×ULN
Subject is a candidate to receive CAPOX (Cohort A, C) or SOX (Cohort B, D) and/or nivolumab (Cohort C, D).
Ability to take oral medications.
Measurable disease, or non-measurable, but evaluable disease, according to RECIST v1.1.

### Exclusion Criteria

Subjects are excluded from the study if any of the following criteria apply.

### Disease Related

Untreated or symptomatic CNS metastases and leptomeningeal disease.
Subjects with asymptomatic CNS metastases are eligible if clinically stable for at least 4 weeks and do not require intervention (including use of corticosteroids).
Subjects with treated brain metastases are eligible provided the following criteria are met:
   Definitive therapy was completed at least 2 weeks prior to the first planned dose of study treatment (stereotactic radiosurgery at least 7 days prior to first planned dose of study treatment).
   At least 7 days prior to first dose of study treatment: any CNS disease is clinically stable, subject is off steroids for CNS disease (unless steroids are indicated for a reason unrelated to CNS disease), and subject is off or on stable doses of anti-epileptic drugs.

### Other Medical Conditions

Known positive HER2 status (as defined by a positive IHC test of 3+ or IHC 2+ with positive Fluorescence in situ hybridization [FISH])

Impaired cardiac function or clinically significant cardiac disease including: unstable angina within 6 months prior to first dose of study treatment, acute myocardial infarction <6 months prior to first dose of study treatment, New York Heart Association (NYHA) class II-IV congestive heart failure, uncontrolled hypertension (defined as an average systolic blood pressure >160 mmHg or diastolic >100 mmHg despite optimal treatment (measured following European Society for Hypertension/European Society of Cardiology [ESH/ESC] 2013 guidelines), uncontrolled cardiac arrhythmias requiring anti-arrhythmic therapy other than beta blockers or digoxin, active coronary artery disease or corrected QT interval (QTc) ≥470.

### Peripheral Sensory Neuropathy Grade 2 or Higher.

Active infection requiring systemic treatment or any uncontrolled infection within 14 days prior to first dose of study treatment.

Known human immunodeficiency virus (HIV) infection with CD4+ T-cell (CD4+) counts <350 cells/μL, hepatitis C infection (subjects with hepatitis C that achieve a sustained virologic response following antiviral therapy are allowed), or hepatitis B infection (subjects with hepatitis B surface antigen [SAg] or core antibody) that achieve sustained virologic response with antiviral therapy directed at hepatitis B are allowed.

History of interstitial lung disease.

Evidence of, or recent (within 6 months) history of, corneal defects, corneal ulcerations, keratitis, or keratoconus, history of corneal transplant, or other known abnormalities of the cornea that may pose an increased risk of developing a corneal ulcer. Recent (within 6 months) corneal surgery or ophthalmic laser treatment.

Evidence of any ongoing ophthalmological abnormalities or symptoms that are acute (within 4 weeks) or actively progressing.

Unwillingness to avoid use of contact lenses during study treatment.

Gastrointestinal (GI) tract disease causing the inability to take oral medication, malabsorption syndrome, requirement for IV alimentation, uncontrolled inflammatory GI disease (e.g., Crohn's disease, ulcerative colitis).

History of other malignancy within the past 2 years, with the following exceptions:
Curatively treated non-melanoma skin malignancy
Cervical cancer in situ
Curatively treated uterine cancer stage I
Curatively treated ductal or lobular breast carcinoma in situ and not currently receiving any systemic therapy
Localized prostate cancer that has been treated surgically with curative intent and presumed cured

Cohorts C and D only:
History of solid organ transplantation.
Active autoimmune disease that has required systemic treatment (except replacement therapy) within the past 2 years or any other diseases requiring immunosuppressive therapy while on study.

### Prior/Concomitant Therapy

Prior treatment for metastatic or unresectable disease.
Prior adjuvant or neo-adjuvant therapy for localized disease is allowed, provided it has been completed more than 6 months prior to the first dose of study treatment
Palliative radiotherapy is allowed, provided it has been completed more than 14 days prior to the first dose of study treatment
All treatment-related toxicity needs to be resolved to grade ≥1 prior to the first dose of study treatment, with the exception of alopecia or toxicities considered irreversible (defined as having been present and stable for >21 days) which are not otherwise described in the exclusion criteria

Prior treatment with any selective inhibitor of the FGF-FGFR pathway.

Major surgical procedures within 28 days prior to first dose of study treatment.

Minor surgery requiring local/epidural anesthesia must be completed more than 72 hours before first dose of study treatment. In all cases, the subject must be sufficiently recovered and stable before treatment administration.

Use of oral coumarin-derivative anticoagulants.

Cohorts B and D only: Recent use (in the past 24 weeks) of flucytosine, an antifungal drug.

Cohorts C and D only: Immunosuppressive doses of systemic medications of >10 mg/day or prednisone or equivalent must be discontinued at least 2 weeks before the first dose of study drug. Short courses of high dose corticosteroids and/or continuous low dose prednisone (<10 mg/day) are allowed. In addition, inhaled, intranasal, intraocular, and/or joint injections of corticosteroids are allowed. Subjects who experienced severe, life-threatening or recurrent (grade 2 or higher) immune-mediated adverse events or infusion-related reactions including those that led to permanent discontinuation while on treatment with immune oncology agents.

### Prior/Concurrent Clinical Study Experience

Currently receiving treatment in another investigational device or drug study within 28 days of enrollment during this clinical study. Other investigational procedures while participating in this study are excluded.

### Other Exclusions

Female subjects of childbearing potential unwilling to use protocol specified method of contraception during treatment and for an additional 9 months after the last dose of protocol-mandated treatment.

Female subjects who are breastfeeding or who plan to breastfeed while on study through 5 months after the last dose of protocol-mandated treatment.

Female subjects planning to become pregnant while on study through 9 months after the last dose of protocol-mandated treatment.

Female subjects of childbearing potential with a positive pregnancy test assessed at screening and within 72 hours prior to first dose of study treatment by a highly sensitive urine or serum pregnancy test.

Male subjects with a female partner of childbearing potential who are unwilling to practice sexual abstinence (refrain from heterosexual intercourse) or use contraception during treatment and for an additional 6 months after the last dose of protocol-mandated therapy.

Male subjects unwilling to abstain from donating sperm during treatment and for an additional 6 months after the last dose of protocol-mandated therapy.

Known allergy, hypersensitivity or contraindication to components of the bemarituzumab formulation including polysorbate or to platinum-containing medications, capecitabine (including subjects with complete dihydropyrimidine dehydrogenase [DPD] deficiency; Cohort A, C), S-1 (including subjects with complete DPD deficiency; Cohort B, D), or nivolumab (Cohort C, D).

Subject likely to not be available to complete all protocol-required study visits or procedures, and/or to comply with all required study procedures (e.g., Clinical Outcome Assessments) to the best of the subject and investigator's knowledge.

History or evidence of (any other clinically significant disorder, condition or disease (with the exception of those outlined above) that, in the opinion of the investigator or Amgen physician, if consulted, would pose a risk to subject safety or interfere with the study evaluation, procedures or completion.

### Bemarituzumab, CAPOX or SOX, and Nivolumab

Information on bemarituzumab, CAPOX, SOX, and nivolumab in the study, including dosing and dosing instructions are shown in Tables 4 and 5 below:

TABLE 4
Investigational Product: Bemarituzumab and Administration Thereof

| | |
|---|---|
| Dosage Formulation | Bemarituzumab is supplied as a sterile, aqueous, |
| | colorless to slightly yellowish, pyrogen-free solution |
| | supplied in single-use glass vials. The composition of |
| | the drug product contains 20 mg/mL active ingredient |
| | (supplied as a 20 mL vial [400 mg bemarituzumab |
| | per vial]), L-histidine, sucrose, and polysorbate 20 at |
| | pH 6.0. The final drug product will be provided as a |
| | solution which should be stored refrigerated (2° C. to |
| | 8° C.), protected from light, and will be diluted for |
| | administration according to the instructions provided |
| | to the site. |
| Dosage Level(s) | 15 mg/kg IV Q3W with an additional dose of |
| | 7.5 mg/kg on cycle 1 day 8 OR 22 mg/kg Q3W with |
| | an additional 11 mg/kg on cycle 1 day 8. Each cycle |
| | is 21 days. |
| Route of Administration | IV Infusion |
| Accountability | The planned dose, quantity administered, start |
| | date/time, stop date/time, lot number of |
| | investigational product, reason total quantity changed |
| | or administration withheld, reason for administration |
| | delay, and reason for infusion interruption are to be |
| | recorded on each subject's eCRF(s). |
| Dosing Instructions | Bemarituzumab will be administered as an IV |
| | infusion, under medical supervision, over |
| | approximately 30-minute (±10 minutes) via a |
| | peripheral vein or central venous catheter. The IV |
| | administration set for bemarituzumab infusion must |
| | contain a 0.22 μm in-line filter. |

eCRF = electronic Case Report Form;
IV = intravenous;
Q3W = every 3 weeks

TABLE 5
Dosage Formulation and Administration for CAPOX, SOX, and Nivolumab

| | Oxaliplatin | Capecitabine | S-1 | Nivolumab |
|---|---|---|---|---|
| Dosage | Sponsor designee will | Sponsor designee | Sponsor designee | Sponsor |
| Formulation | provide oxaliplatin for | will provide | will provide S-1 in | designee will |
| | injection: 50 mg | capecitabine 150, | capsules in one of | provide |
| | (5 mg/mL) or 100 mg | 300, or 500 mg | two strengths: | nivolumab for |
| | (5 mg/mL) in a single | tablets. | containing 20 mg | injection: |
| | dose vial. | | tegafur, 5.8 mg | 20 mg/2 mL, |
| | | | gimeracil, and | 40 mg/4 mL, |
| | | | 19.6 mg oteracil or | 100 mg/10 |
| | | | 25 mg tegafur, | mL, or |
| | | | 7.25 mg gimeracil, | 240 mg/24 mL |
| | | | and 24.5 mg | solution in a |
| | | | oteracil. | single-dose |
| | | | | vial. |
| Route of | IV infusion | Oral | Oral | IV infusion |
| Administration | | | | |
| Accountability | The planned dose, total volume of preparation, lot number (unless unable to be | | | |
| | recorded due to local prescribing procedure in countries where standard of care is | | | |
| | deemed non-investigational) concentration of preparation, quantity administered, | | | |
| | start date/time, stop date/time, reason total quantity changed, or administration | | | |
| | withheld, reason for administration delay, and reason for infusion interruption are to | | | |
| | be recorded on each subject's eCRF(s) | | | |
| Dosing | Oxaliplatin 130 mg/m² | Capecitabine | S-1 40 mg/m² | Nivolumab |
| Instructions | IV will be administered | 1000 mg/m² orally | orally twice daily | 360 mg will be |
| | on day 1 of every | twice daily, with | after a meal, | administered |
| | 21 day cycle under | water within 30 | starting on day 1 | on day 1 of |
| | medical supervision | min after a meal, | in the evening | every 21-day |
| | over 120 minutes. | starting on day 1 | through the | cycle under |
| | Infusion can be | in the evening | morning of day | medical |
| | extended according to | through the | 15. The same | supervision |
| | regional prescribing | morning of day | BSA formula | over |
| | information. Dose | 15. The same | should be used | approximately |
| | should be calculated | BSA formula | during the | 30 minutes |
| | before each | should be used | treatment period. | (±10 minutes). |
| | administration per | during the | Dose should be | |
| | institutional practice. | treatment period. | calculated before | |
| | | Dose should be | dispensing S-1. | |
| | | calculated before | | |
| | | dispensing | | |
| | | Capecitabine. | | |

eCRF = electronic Case Report Form;
BSA = body surface area;
IV = intravenous

### Dose Level Determination

The study will explore the dose of bemarituzumab in combination with other anti-cancer therapies. The study DLT period is 21 days following the first dose. For a given cohort, once 3 to 6 subjects are enrolled at a certain dose level are followed for safety for 21 days, a DLRT meeting is convened. The DLRT will evaluate all available safety, laboratory, and PK as well as rules generated from a BOIN design (Liu and Yuan, 2015; Yuan et al, 2016) to guide their dose finding recommendations. The DLRT may recommend escalation to the next planned dose level, continuation at the current dose level, de-escalation to a lower dose level, or termination of the cohort. The BOIN escalation/de-escalation guideline for each cohort is derived with a target toxicity rate of 0.25. Decisions to dose escalate will take into account all available safety data from all cohorts and include available data from beyond the 21-day DLT window. After receiving the DLRT recommendation, the team will render a final decision and will issue a written notification of the dose change decision to investigators.

The DLRT will use guidelines for each cohort based on a BOIN design as described below:
Subjects in a given cohort are treated at dose level 1.
To assign a dose to the next cohort of subjects, the guidelines in Table 6 will be used, nothing the following:
“Eliminate” means that the current and higher doses are eliminated from the cohort to prevent treating any future subjects at these doses because they are overly toxic.
When a dose is eliminated, de-escalation to the next lower dose level occurs. When the lowest dose is eliminated, the cohort is stopped for safety.
If none of the actions (i.e., escalation, de-escalation, or elimination) is triggered, new subjects are treated at the current dose.
If the current dose is the lowest dose and the rule indicates dose de-escalation, new subjects are treated at the lowest dose unless the number of DLTs reaches the elimination boundary, at which point the trial is stopped for safety
If the current dose is the highest dose and the rule indicates dose escalation, treat the new patients at the highest dose.

Step 2 is repeated until a minimum of 6 subjects and a maximum 16 is reached for a dose level of interest, and the decision according to Table 6 is to stay at the current dose.

TABLE 6
Dose Escalation/De-Escalation Rules Based on the BOIN Design

| | # of evaluable subjects | | | | | | | | | | | | | | | |
|---|---|---|---|---|---|---|---|---|---|---|---|---|---|---|---|---|
| | 1 | 2 | 3 | 4 | 5 | 6 | 7 | 8 | 9 | 10 | 11 | 12 | 13 | 14 | 15 | 16 |
| Escalate if # of | 0 | 0 | 0 | 0 | 0 | 1 | 1 | 1 | 1 | 1 | 2 | 2 | 2 | 2 | 2 | 3 |
| DLT≤ | | | | | | | | | | | | | | | | |
| Deescalate if # | 1 | 1 | 1 | 2 | 2 | 2 | 3 | 3 | 3 | 3 | 4 | 4 | 4 | 5 | 5 | 5 |
| of DLT≥ | | | | | | | | | | | | | | | | |
| Eliminate if # | NA | NA | 3 | 3 | 3 | 4 | 4 | 4 | 5 | 5 | 6 | 6 | 6 | 7 | 7 | 7 |
| of DLT≥ | | | | | | | | | | | | | | | | |

BOIN = Bayesian optimal interval;
DLT = Dose Limiting Toxicities

### Dose-Limiting Toxicities

Dose-limiting toxicities during dose exploration are defined as any of the following adverse events considered by the investigator to be related to study drug:
Grade 4 neutropenia of any duration
Febrile neutropenia
Grade 4 thrombocytopenia
Grade 3 thrombocytopenia with grade 2 bleeding or lasting >7 days
Grade 4 anemia
Grade 5 toxicity (e.g., death not due to disease progression)
Any Grade 3 ophthalmologic adverse event that does not resolve within 7 days
Any Grade 4 ophthalmologic adverse event
Any Grade 4 laboratory value
Any Grade 4 vomiting or diarrhea
Grade 3 vomiting or Grade 3 diarrhea lasting more than 3 days despite optimal medical support
Grade 3 nausea lasting 3 days or more despite optimal medical support
Grade 3 fatigue lasting 1 week or longer

Any subject meeting the criteria for Hy's Law case (i.e., severe drug-induced liver injury [DILI]) will be considered a DLT. A Hy's Law case is defined as: AST or ALT values of ≥3×ULN AND with serum TBIL level of >2×ULN or international normalized ratio (INR) >1.5 without signs of cholestasis and with no other clear alternative reason to explain the observed liver related laboratory abnormalities (see Section 11.7 for further explanation of Hy's law case and Management of Hepatic Function).

Any other grade ≥3 adverse event with the following exceptions:
DLT Exemption: Asymptomatic Grade 3 electrolyte abnormalities that last <72 hours, are not clinically complicated, and resolve spontaneously or respond to medical interventions.
DLT Exemption: Other select lab abnormalities that do not appear to be clinically relevant or harmful to the subject (e.g., grade 3 lymphopenia, grade 3 hypoalbuminemia), and/or can be corrected with replacement or modifications.
DLT Exemption: A transient (resolving to grade 1 within 6 hours of onset) grade 3 infusion-related adverse event.
DLT Exemption: Any grade 3 endocrinopathy that is adequately controlled by hormonal replacement.

Subjects enrolled in dose exploration may be replaced if they are not evaluable for a DLT (e.g., a subject did not receive a planned study treatment [at least 100% of planned doses for IV agents and at least 80% of planned doses for oral agents]), or ended the study treatment before completion of DLT evaluation period for a reason other than experiencing a DLT. The replaced subject may continue on study at the Investigator's discretion and after discussion with the Medical Monitor.

If an adverse event is clearly attributable to any component of the combination regimen (based on its known safety profile) and does not exceed the expected severity, this specific adverse event may be considered exempt from being a DLT.

### Bemarituzumab Dosage Adjustments, Delays, Rules for Withholding or Restarting, Permanent Discontinuation

Bemarituzumab doses may be held for bemarituzumab-related adverse events following the guidelines outlined in Table 7.1. The reason for dose delay of bemarituzumab is to be recorded on each subject's CRFs.

After cycle 1, the bemarituzumab dose should be recalculated only if the weight changes >10% from the cycle 1 day 1 (C1D1) weight. If the dose is recalculated due to a >10% weight change from C1D1, the weight used for the recalculated dose should function as the new baseline for subsequent evaluation of dose recalculations.

If dose reductions or interruptions that do not fall within these guidelines are being considered by the investigator, these will require discussion with the Sponsor or designee. Cycles may be delayed to manage toxicity. Cycle delays of longer than 21 days should be discussed with the medical monitor prior to re-initiation.

Corneal events: any subject with a corneal event which occurs within 100 days of last receiving a dose of bemarituzumab, regardless if deemed related or not related to bemarituzumab, should be evaluated by an ophthalmologist. Any subject who reports pain or irritation of the eye or change in vision should be evaluated by an ophthalmologist. Tables 7.1, 7.2, and 7.3 provide additional guidance.

TABLE 7.1
Bemarituzumab Dose Modification Guidelines for Adverse Events

| | | | | |
|---|---|---|---|---|
| | | | | Permanent |
| Grade | Interruption/Delay | Specific Management | Re-start Guidance | Discontinuation |

Corneal Toxicity^{a}

| | | | | |
|---|---|---|---|---|
| 1 | n/a | n/a | n/a | n/a |
| 2 | Clinical judgement | Refer to ophthalmologist for | If held/delayed, | Discontinue if |
| | required. If subject | evaluation | resume at full dose if | grade 3 or higher |
| | has not been seen by | Treatment as deemed | event has improved | event recurs with |
| | ophthalmologist for | appropriate by the | to grade ≤1 and best | restart of the drug. |
| | the Grade 2 event, | ophthalmologist. | visual acuity is | |
| | treatment should | Bemarituzumab may be | decreased no more | |
| | either be | continued or held, based on | than 2 Snellen chart | |
| | held/delayed until | ophthalmologic findings as | lines from known | |
| | ophthalmologic | long as best visual acuity is | baseline. | |
| | evaluation or, if not | decreased no more than | | |
| | associated with a | 2 Snellen chart lines from | | |
| | change in vision, | known baseline. | | |
| | subject may receive | | | |
| | a maximum of 1 | | | |
| | dose prior to | | | |
| | ophthalmologic | | | |
| | evaluation. | | | |
| 3 | Immediate | Refer to ophthalmologist for | Resume at full dose | Discontinue |
| | interruption/delay | evaluation urgently. | if event has | treatment if event |
| | until event has | Treatment as deemed | improved to | does not improve |
| | improved to | appropriate by the | grade ≤1. | to grade ≤1 |
| | grade ≤1 | ophthalmologist. | | |
| | | Close follow up by | | Discontinue if |
| | | ophthalmologist until event | | grade 3 or higher |
| | | has improved to grade ≤1. | | event recurs with |
| | | | | restart of the drug. |
| 4 | n/a | Refer to ophthalmologist for | n/a | Discontinue |
| | | evaluation urgently. | | bemarituzumab |
| | | Treatment as deemed | | |
| | | appropriate by the | | |
| | | ophthalmologist. | | |
| | | Close follow up by | | |
| | | ophthalmologist until event | | |
| | | has improved to grade ≤1. | | |

TABLE 7.2
Bemarituzumab Dose Modification Guidelines for Adverse Events

| | | | | |
|---|---|---|---|---|
| CTCAE | Interruption/ | | | Permanent |
| Grade | Delay | Specific Management | Re-start guidance | Discontinuation |

Infusion-related Reaction

| | | | | |
|---|---|---|---|---|
| 1 | Decrease rate | Increase monitoring of vital signs | n/a | n/a |
| | of infusion by | as medically indicated until the | | |
| | 50% | subject is deemed medically | | |
| | | stable. | | |
| | | Treat per institutional guidelines. | | |
| 2 | Immediate | Increase monitoring of vital signs | After all symptoms have | For subjects who |
| | interruption/ | as medically indicated until the | resolved: administer | develop grade 2 |
| | delay until event | subject is deemed medically | premedication (e.g., | infusion-related |
| | has improved | stable. | antihistamines, | reaction despite |
| | to grade ≤1 | Treat per institutional guidelines. | corticosteroids, and | adequate |
| | | Additional appropriate medical | acetaminophen) | premedication, |
| | | therapy may include but is not | 1.5 hours (±30 | permanently |
| | | limited to IV fluids, | minutes) prior to | discontinue |
| | | antihistamines, NSAIDs, | infusion using | bemarituzumab. |
| | | acetaminophen, and narcotics. | institutional standards | |
| | | | resume bemarituzumab | |
| | | | at reduced infusion | |
| | | | rate (50% or less of | |
| | | | standard rate) | |
| 3 or 4 | Immediate | Increase monitoring of vital signs | n/a | Permanently |
| | interruption | as medically indicated until the | | discontinue |
| | | subject is deemed medically | | bemarituzumab |
| | | stable. | | |
| | | Hospitalization may be indicated. | | |
| | | Treat per institutional guidelines. | | |
| | | Additional appropriate medical | | |
| | | therapy may include but is not | | |
| | | limited to IV fluids, | | |
| | | antihistamines, NSAIDs, | | |
| | | acetaminophen, narcotics, | | |
| | | oxygen, vasopressors, | | |
| | | corticosteroids, and epinephrine. | | |
| | | In cases of anaphylaxis, | | |
| | | epinephrine should be used | | |
| | | immediately. | | |

TABLE 7.3
Bemarituzumab Dose Modification Guidelines for Adverse Events

| | | | | |
|---|---|---|---|---|
| CTCAE | | Specific | | Permanent |
| Grade | Interruption/Delay | Management | Re-start guidance | Discontinuation |

All other bemarituzumab-related adverse events

| | | | | |
|---|---|---|---|---|
| 1 or 2 | n/a | Treat per | n/a | n/a |
| | | institutional | | |
| | | guidelines. | | |
| 3 (first | Delay or miss dose | Treat per | If recovery to | n/a |
| occurrence) | until recovery to | institutional | baseline or grade 1, | |
| | baseline or grade 1 | guidelines. | may resume at full | |
| | | | dose | |
| 3 (second | Delay or miss dose | Treat per | Resume at full dose if | n/a |
| occurrence) | until recovery to | institutional | event has improved to | |
| | baseline or grade 1 | guidelines. | grade ≤1 | |
| Grade 3 (third | Immediate | Treat per | n/a | Permanently |
| occurrence) | interruption | institutional | | discontinue |
| Grade 3 which | | guidelines. | | bemarituzumab |
| does not | | | | |
| recover to | | | | |
| baseline or | | | | |
| grade 1 within | | | | |
| 21-days of | | | | |
| onset event | | | | |
| Any grade 4 | | | | |

CTCAE = Common Terminology Criteria for Adverse Events;
IV = intravenous;
N/A = not applicable;
NSAIDs = nonsteroidal anti-inflammatory drugs
^{a} Ocular adverse events that are not corneal toxicity should follow dose modifications for all other bemarituzumab-related adverse events.

### CAPOX Dosage Adjustments, Delays, Rules for Withholding or Restarting, Permanent Discontinuation

### Oxaliplatin

Dose modifications of oxaliplatin are permitted according to institutional standards or local package inserts.

### Neurotoxicity

For grade 2 peripheral sensory neuropathy (moderate paresthesia or dysthesia), or limiting instrumental activities of daily living, oxaliplatin should be skipped. When toxicity resolves to grade 1, resume oxaliplatin to 75% of initial dose. If oxaliplatin is skipped for 6 weeks (2 consecutive doses) for neurologic toxicity, discontinue oxaliplatin.

### Renal Impairment

For normal renal function or mild to moderate renal impairment (CrCl>50 mL/min), the full dose of oxaliplatin can be administered. For severe renal impairment, the oxaliplatin dose should be reduced to 75% of initial dose.

### Hematologic Toxicity

For grade 2 or grade 3 thrombocytopenia, oxaliplatin should be reduced to 75% of initial dose. For grade 4 thrombocytopenia, the dose should be reduced to 50% of initial dose. For grade 3 or grade 4 neutropenia or febrile neutropenia, the oxaliplatin dose should be reduced to 75% of initial dose.

### Allergic Reaction

If a subject experiences grade 1 or 2 allergic reaction to oxaliplatin, premedication should be given according to institutional practice prior to subsequent further study drug administration. If a grade 1 or 2 allergic reaction persists into the next cycle, escalation of the appropriate premedication should be done according to institutional practice prior to administration of oxaliplatin. For subjects experiencing grade 3 or 4 allergic reactions, treatment with oxaliplatin should be discontinued.

### Capecitabine

Dose modifications of capecitabine are permitted according to institutional standards or local package inserts.

General dose modification guidelines for capecitabine are presented in Table 8. Toxicity due to capecitabine administration may be managed by symptomatic treatment, dose interruption, and adjustment of capecitabine dose. Once the dose has been reduced, it should not be increased at a later time. The only exception to this practice will be in the case of nausea/vomiting. If grade 2 or higher nausea and/or vomiting occur despite adequate antiemetic therapy, the chemotherapy dose should be reduced by 25% for the next dose. Adequate antiemetic therapy should include at least 2 modalities of medications (e.g., 5HT-3 receptor antagonists, NK1 receptor antagonist). If tolerated, an increase back to a 100% dose may be allowed at the investigator's discretion. Chemotherapy cycles may be delayed to manage toxicity. Cycle delays of longer than 21 days should be discussed with the Medical Monitor prior to re-initiation of treatment.

Doses of capecitabine omitted for toxicity are not replaced or restored; instead the subject should resume the planned treatment cycles. There is a drug interaction risk of phenytoin and capecitabine. The dose of phenytoin may need to be reduced when co administered concomitantly with capecitabine as phenytoin levels may be increased due to inhibition of CYP2C9 by capecitabine, resulting in increase in toxic levels of phenytoin.

Dihydropyrimidine dehydrogenase deficiency, an enzyme deficiency, can first manifest as severe, life-threatening reaction to fluoropyrimidines. Evaluation for DPD deficiency should be considered if the following events occur after capecitabine administration: severe diarrhea, mucositis, neutropenia, or neurotoxicity.

TABLE 8
Recommended Dose Modification for Capecitabine

| | | Dose Adjustment for Next |
|---|---|---|
| | | Treatment (% of Starting |
| Toxicity Grade^{a} | During course of Therapy | Dose) |
| Grade 1 | Maintain dose level | Maintain dose level |

Grade 2

| | | |
|---|---|---|
| First appearance | Interrupt until resolved to grade 0 to 1 | 100% |
| Second appearance | | 75% |
| Third appearance | | 50% |
| Fourth appearance | Discontinue treatment permanently | — |

Grade 3

| | | |
|---|---|---|
| First appearance | Interrupt until resolved to grade 0 to 1 | 75% |
| Second appearance | | 50% |
| Third appearance | Discontinue Treatment permanently | — |

Grade 4

| | | |
|---|---|---|
| First appearance | Discontinue permanently OR if | 50% |
| | physician deems it to be in subject's | |
| | best interest to continue, interrupt until | |
| | resolved to grade 0 to 1 | |

Source: XELODA US Prescribing Information 2021
^{a}According to the National Cancer Institute of Canada Clinical Trial Group (NCIC CTG) Common Toxicity Criteria (version 1.0) or the Common Terminology Criteria for Adverse Events (CTCAE) of the Cancer Therapy Evaluation Program, US National Cancer Institute (version 4.0).

### SOX Dosage Adjustments, Delays, Rules for Withholding or Restarting, Permanent Discontinuation

### S-1

Dose modifications of S-1 are permitted according to institutional standards or local package inserts.

General dose modification guidelines for S-1 are presented in Table 9. Toxicity due to S-1 administration may be managed by symptomatic treatment, dose interruption, and adjustment of S-1 dose. Once the dose has been reduced, it should not be increased at a later time. The only exception to this practice will be in the case of nausea/vomiting. If grade ≥2 nausea and/or vomiting occur despite antiemetic therapy, the chemotherapy dose should be reduced for the next dose. If tolerated, an increase back to a standard dose may be allowed at the investigator's discretion. If a drug rest therapeutically needs to be shortened, it should be implemented after confirming that no drug-induced abnormalities in laboratory findings (hematologic tests, liver and renal function tests) and no gastrointestinal symptoms occur, i.e., the drug is not problematic in terms of safety. A minimum drug rest period of 7 days must be provided. Cycle delays of longer than 21 days should be discussed with the Medical Monitor prior to re-initiation of treatment. Doses of S-1 omitted for toxicity are not replaced or restored; instead the subject should resume the planned treatment cycles.

There is a drug interaction risk of phenytoin and S-1. The dose of phenytoin may need to be reduced when co-administered concomitantly with S-1 as phenytoin levels may be increased due to inhibition of CYP2C9 by S-1, resulting in increase in toxic levels of phenytoin.

Dihydropyrimidine dehydrogenase (DPD) deficiency, an enzyme deficiency, can first manifest as severe, life-threatening reaction to fluoropyrimidines. Evaluation for DPD deficiency should be considered if the following events occur after S-1 administration: severe diarrhea, mucositis, neutropenia, or neurotoxicity.

In the event that oxaliplatin administration is discontinued for any reason prior to disease progression, S-1 may continue until disease progression or unacceptable toxicity. If S-1 is permanently discontinued, then oxaliplatin must be discontinued. Table 9 shows the recommended dose modification for S-1. Further details can be found in regional prescribing information. Institutional dose reduction guidelines can also be applied.

TABLE 9
Recommended Dose Modification for S-1

| Standard Dose by body | | | | | |
|---|---|---|---|---|---|
| surface area | | Dose Reduction 1 by body | | Dose Reduction 2 by body | |
| | Dose | surface area | | surface area | |
| Body surface | (tegafur | Body surface | Dose (tegafur | Body surface | Dose (tegafur |
| area (m²) | equivalent) | area (m²) | equivalent) | area (m²) | equivalent) |
| <1.25 | 40 mg BID | <1.25 | Drug rest | <1.25 | N/A |
| 1.25-<1.5 | 50 mg BID | 1.25-<1.5 | 40 mg BID | 1.25-<1.5 | Drug rest |
| ≥1.5 | 60 mg BID | ≥1.5 | 50 mg BID | ≥1.5 | 40 mg BID |

BID = twice a day,
N/A = not applicable
Source: TS-ONE Japan Prescribing Information 2021

### Nivolumab Dosage Adjustments, Delays, Rules for Withholding or Restarting, Permanent Discontinuation

No dose reduction for nivolumab is recommended. In general, nivolumab should be held for grade 3 immune-mediated adverse reactions, and permanently discontinued for life-threatening (grade 4) immune-mediated adverse events, recurrent severe (grade 3) immune-mediated reactions that required systemic immunosuppressive treatment, or an inability to reduce corticosteroid dose to 10 mg or less of prednisone or equivalent per day within 12 weeks of initiating steroids. Additional information is shown in Table 10. If nivolumab is discontinued for toxicity, chemotherapy and bemarituzumab may be continued. If all components of chemotherapy are discontinued, nivolumab may be continued. If any component of nivolumab is delayed or held in the first 3 cycles, bemarituzumab should continue as scheduled. If any component of nivolumab is delayed for up to 7 days after the first 3 cycles, bemarituzumab should be delayed as well to align with nivolumab dosing. If any component of nivolumab is held for more than 7 days or is discontinued after the first 3 cycles, bemarituzumab should continue per schedule.

The reason for discontinuation of nivolumab is to be recorded on each subject's eCRF(s).

TABLE 10
Nivolumab Dose Modifications

| Adverse | | |
|---|---|---|
| Reaction | Severity | Dosage Modification |
| Pneumonitis | Grade 2 | Withhold^{a} |
| | Grade 3 or 4 | Permanently discontinue |
| Colitis | Grade 2 or 3 | Withhold^{a} |
| | Grade 4 | Permanently discontinue |
| Hepatitis with no | AST/ALT increases to >3 and ≤8 times ULN or | Withhold^{a} |
| tumor | total bilirubin increases to >1.5 and ≤3 times ULN | |
| involvement of | AST/ALT increases to >8 times ULN or total | Permanently discontinue |
| the liver | bilirubin increases to >3 times ULN | |
| Hepatitis with | Baseline AST/ALT is >1 and ≤3 times ULN and | Withhold^{a} |
| tumor | increases to >5 and ≤10 times ULN or baseline | |
| involvement of | AST/ALT is >3 and ≤5 times ULN and increases | |
| the liver^{b} | to >8 and ≤10 times ULN | |
| | AST/ALT increases to >10 times ULN or total | Permanently discontinue |
| | bilirubin increases to >3 times ULN | |
| Endocrinopathies^{c} | Grade 3 or 4 | Withhold until clinically |
| | | stable or permanently |
| | | discontinue depending on |
| | | severity |
| Nephritis with | Grade 2 or 3 increased blood creatinine | Withhold^{a} |
| renal dysfunction | Grade 4 increased blood creatinine | Permanently discontinue |
| Exfoliative | Suspected SJS, TEN, or DRESS | Withhold^{a} |
| dermatologic | Confirmed SJS, TEN, or DRESS | Permanently discontinue |
| conditions | | |
| Myocarditis | Grade 2, 3, or 4 | Permanently discontinue |
| Neurological | Grade 2 | Withhold^{a} |
| toxicities | Grade 3 or 4 | Permanently discontinue |
| Infusion-related | Grade 1 or 2 | Interrupt or slow the rate of |
| reactions | | infusion |
| | Grade 3 or 4 | Permanently discontinue |

Source: OPDIVO U.S. Prescribing Information, 2021
ALT = alanine aminotransferase;
AST = aspartate aminotransferase;
DRESS = Drug Rash with Eosinophilia and Systemic Symptoms;
SJS = Stevens Johnson Syndrome;
TEN = toxic epidermal necrolysis; ULN = upper limit of normal
^{a}Resume in subjects with complete or partial resolution (Grade 0 to 1) after corticosteroid taper. Permanently discontinue if no complete or partial resolution within 12 weeks of last dose or inability to reduce prednisone to 10 mg per day (or equivalent) or less within 12 weeks of initiating steroids.
^{b}If AST and ALT are less than or equal to ULN at baseline, withhold or permanently discontinue OPDIVO based on recommendations for hepatitis with no liver involvement. Other causes of hepatitis should be excluded before attributing the abnormalities to nivolumab.
^{c}Depending on clinical severity, consider withholding for Grade 2 endocrinopathy until symptom improvement with hormone replacement. Resume once acute symptoms have resolved.

### Treatment Compliance

When subjects are dosed at a site, they will receive bemarituzumab, oxaliplatin, and nivolumab (where applicable), directly from the investigator or designee, under medical supervision. The date and time of each dose administered in the clinic will be recorded in the source documents and recorded in the eCRF.

Capecitabine and S-1 are administered by the subject at home. Compliance will be assessed by direct questioning, review of diary, and counting returned study medication. The following procedures will be employed to assure appropriate drug accountability. Drug accountability will be emphasized at the start-up meeting. Drug accountability will be monitored throughout the study. Each subject will be instructed to return all study drug packaging and unused material to the study site at each visit. The study site will keep a record of all study drug dispensed to and returned by the subjects throughout the study. Study site personnel will return all unused study drug for all subjects. Each subject will be instructed to keep a study diary to document that he/she is taking the study drug correctly.

The subject must take ≥80% to ≥100% of the intended dose to be deemed compliant with administration of capecitabine and S-1. Similarly, a subject may be considered noncompliant if he/she is judged by the investigator to have intentionally or repeatedly taken less or more than the prescribed amount (i.e., <80% or >100%). Potential discontinuation of a subject due to study drug noncompliance will be discussed between the investigator and the Medical Monitor before the final determination is made to discontinue the subject.

### Prior and Concomitant Treatment

### Prior Treatment

All prior medications must be recorded in the designated eCRF forms. Prior therapies that were being taken/used from 3 months prior to enrollment will be collected.

For prior therapies for gastric or gastroesophageal junction cancer, therapy name, setting, dose, unit, frequency, start date, stop date, best response, and reason for discontinuation are collected. For anticancer therapies consisting of multiple individual components, information for each component is collected. For all other prior therapies, therapy name, indication, dose, unit, frequency, route, start date, and stop date are collected.

### Concomitant Treatment

Throughout the study, investigators may prescribe any concomitant medications or treatments deemed necessary to provide adequate supportive care.

To mitigate the risk of corneal toxicities, prophylactic use of ocular lubricants and eyelid hygiene is recommended. Ocular lubricants (e.g., preservative free artificial tears) should be self-administered 3 times daily throughout the treatment period and for 30 days after the last dose. They may be polyvinyl alcohol- or liquid polyol based. If preservative free is not available, formulations with preservatives are allowed. Methylcellulose-based lubricants should not be used. Viscous lubricants which can cause blurriness should be avoided.

Concomitant therapies are to be collected from informed consent through the end of SFU, with the exception of ophthalmologic and anticancer therapies, which will be collected through LTFU. For concomitant therapies, including vaccines, therapy name, indication, dose, unit, frequency, route, start date, and stop date are collected.

For subsequent therapies taken for gastric or gastroesophageal junction cancer, drug name, start date, and stop date are collected.

### Discontinuation of Study Treatment and Subject Discontinuation/Withdrawal

Subjects have the right to withdraw from investigational product and/or other protocol required therapies, protocol procedures, or the study as a whole at any time and for any reason without prejudice to their future medical care by the physician or at the institution.

The investigator and/or sponsor can decide to withdraw a subject(s) from investigational product, device, and/or other protocol-required therapies, protocol procedures, or the study as a whole at any time prior to study completion for the reasons listed below.

### Discontinuation of Study Treatment

Subjects (or a legally authorized representative) can decline to continue receiving investigational product and/or other protocol-required therapies and/or procedures at any time during the study but continue participation in the study. If this occurs, the investigator is to discuss with the subject the appropriate processes for discontinuation from investigational product or other protocol-required therapies and must discuss with the subject the possibilities for continuation of the Schedule of Activities in Table 3, including different options of follow-up (e.g., in person, by phone/mail, through family/friends, in correspondence/communication with other treating physicians, from the review of medical records) and collection of data, including endpoints, adverse events, and must document this decision in the subject's medical records. Subjects who have discontinued investigational product and/or other protocol-required therapies and/or procedures should not be automatically removed from the study. Whenever safe and feasible, it is imperative that subjects remain on-study to ensure safety surveillance and/or collection of outcome data.

Reasons for early removal from protocol-required investigational product(s) or procedural assessments may include any of the following:
decision by Sponsor
lost to follow-up
death
adverse event
subject request
ineligibility determined
protocol deviation
non-compliance
disease progression
   Radiographic progression (RECIST v1.1)
   clinical progression (investigator's assessment)
requirement for alternative therapy
pregnancy.
   Subject Discontinuation/Withdrawal from the Study

Withdrawal of consent for a study means that the subject does not wish to receive further protocol-required therapies or procedures, and the subject does not wish to or is unable to continue further study participation. Subject data up to withdrawal of consent will be included in the analysis of the study, and where permitted, publicly available data can be included after withdrawal of consent. The investigator is to discuss with the subject appropriate procedures for withdrawal from the study and must document the subject's decision to withdraw in the subject's medical records. Subjects who are withdrawn or removed from treatment or the study will not be replaced. If a subject withdraws from the study, he/she may request destruction of any samples taken and not tested

Reasons for removal of a subject from the study are:
Decision by sponsor
Withdrawal of consent from study
Death
Lost to follow-up.

### Lost to Follow-Up

A subject will be considered lost to follow-up if he or she repeatedly fails to return for scheduled visits and is unable to be contacted by the study site.

The following actions must be taken if a subject fails to return to the clinic for a required study visit:
The site must attempt to contact the subject and reschedule the missed visit as soon as possible and counsel the subject on the importance of maintaining the assigned visit schedule and ascertain whether or not the subject wishes to and/or is able to continue in the study.
In cases in which the subject is deemed lost to follow-up, the investigator or designee must make every effort to regain contact with the subject (where possible, 3 telephone calls and, if necessary, a certified letter to the subject's last known mailing address or local equivalent methods). These contact attempts are to be documented in the subject's medical record.
If the subject continues to be unreachable, he/she will be considered to have withdrawn from the study with a primary reason of lost to follow-up.
For subjects who are lost to follow-up, the investigator should search publicly available records where permitted to ascertain vital status. This ensures that the data set(s) produced as an outcome of the study is/are as comprehensive as possible.

### Efficacy Assessments and Procedures

### Radiographic Imaging Assessment

The extent of disease will be evaluated by contrast-enhanced computed tomography (CT)/magnetic resonance imaging (MRI) according to RECIST v1.1. In order to reduce radiation exposure for subjects, low dose CT should be utilized whenever possible.

### Screening Scans:

The screening scans should be performed within 28 days (the scans may be performed within 31 days) prior to cycle 1 day 1. If there are multiple screening scans, the one closest to the enrollment will be used as baseline.

Radiographic assessment must include CT/MRI of the chest, abdomen and pelvis, as well as assessment of all other known sites of disease per institutional standard.

All subjects with brain metastasis must have MRI of the brain performed. All brain scans for subjects with brain metastasis are required to be MRI unless MRI is contraindicated, and then CT with contrast is acceptable. Brain imaging (MRI or CT) of the brain should be performed if signs or symptoms suggestive of CNS metastases are present.

### Subsequent Scans:

All subsequent scans should be performed in the same manner (e.g., with the same contrast, MRI field strength) as at screening preferably on the same scanner. If the imaging modality must be altered (e.g., unscheduled assessment) consultation with the medical monitor is recommended.

During treatment and follow-up, radiographic imaging of the chest, abdomen, pelvis, as well as all other known sites of disease, will be performed independent of treatment cycle as specified in the Schedule of Activities (see Table 3). Imaging may also be performed more frequently if clinically necessitated at the discretion of the managing physician. Radiographic imaging and tumor assessment will be performed until start of new anticancer therapy, disease progression, death, withdrawal of consent, or end of study, whichever occurs first. Determination of disease response for clinical management and response evaluation will be performed at the clinical sites per RECIST v1.1.

### Safety Assessments

Planned time points for all safety assessments are listed in Table 3.

### Vital Signs

The following measurements must be performed: systolic/diastolic blood pressure, heart rate, respiratory rate, and temperature. Subject must be in a seated position in a rested and calm state for at least 5 minutes before blood pressure assessments are conducted. The position selected for a subject should be the same that is used throughout the study and documented on the vital signs eCRF. The temperature location selected for a subject should be the same that is used throughout the study and documented on the vital signs eCRF.

### Electrocardiograms (ECGs)

Subject must be in supine position in a rested and calm state for at least 5 minutes before ECG assessment is conducted. If the subject is unable to be in the supine position, the subject should be in most recumbent position as possible. The ECG must include the following measurements: Heart rate, QRS, QT, QTc, and PR intervals. The Principal Investigator or designated site physician will review all ECGs. Once signed, the original ECG tracing will be retained with the subject's source documents.

### Clinical Laboratory Assessments

The investigator is responsible for reviewing laboratory test results and recording any clinically relevant changes occurring during the study in the Events eCRF. The investigator must determine whether an abnormal value in an individual study subject represents a clinically significant change from the subject's baseline values. In general, abnormal laboratory findings without clinical significance (based on the investigator's judgment) are not to be recorded as adverse events. However, laboratory value changes that require treatment or adjustment in current therapy are considered adverse events. Where applicable, clinical sequelae (not the laboratory abnormality) are to be recorded as the adverse event.

All protocol-required laboratory assessments must be conducted in accordance with the Schedule of Activities (Table 3).

### Vital Status

Vital status must be obtained for all subjects within the limits of local law. This includes subjects who may have discontinued study visits with or without withdrawing consent and should include interrogation of public databases, if necessary. If deceased, the date and reported cause of death should be obtained.

### Ophthalmologic Examination

Ophthalmologic examinations will be performed according to the Schedule of Activities in Table 3. Ophthalmologic adverse events of any grade occurring up to 100 days after the last dose of bemarituzumab should be reported.

The ophthalmologic examination should include distance corrected visual activity of each eye separately with acuity recorded as the logMAR equivalent, slit lamp examination of the anterior segment, ocular surface staining (e.g., fluorescein), dilated retinal examination or 3 field retinal photographs. Additionally optical coherence tomography (OCT) of the macula is required specifically for subjects diagnosed with retinal pigment epithelium (RPE) detachments.

Ophthalmologic examinations should be performed regardless of dose delays per the schedule of activities. The ophthalmological examination may be repeated at any time, as clinically indicated. After the SFU visit if the subject has any persistent ophthalmologic findings, the assessments should continue until resolution of findings, withdrawal of consent, death, or loss to follow up. Ocular adverse events should be monitored by an ophthalmologist until resolution.

The following measures will be implemented for ophthalmologic adverse events:
Manage as per standard of care
Dose modification and interruption guidelines for the investigator are provided in Table 7.3.

If any clinically significant changes are noted compared to the previous examination or, if the subject has any grade 2 or higher ocular signs or symptoms, the subject should be evaluated.

### Adverse Events

The adverse event grading scale to be used for this study will be the Common Terminology Criteria for Adverse Events (CTCAE) Version v5.0.

The investigator is responsible for ensuring that all adverse events observed by the investigator or reported by the subject that occur after first dose of investigational product(s)/study treatment/protocol-required therapies through the end of SFU visit are reported using the Events eCRF. Adverse events within 28 (+3) days after the last day of dosing investigational product are expected to be recorded.

### Serious Adverse Events

The investigator is responsible for ensuring that all serious adverse events observed by the investigator or reported by the subject that occur after signing of the informed consent through 28 days (+3) after the last day of dosing of investigational product(s) are reported using the Events eCRF. During the pre-screening period, only serious adverse events related to pre-screening procedures will be collected. Serious adverse events suspected to be related to investigational product should be recorded through end of study.

All serious adverse events will be collected, recorded and reported to the sponsor or designee within 24 hours of the investigator's awareness of the event. The investigator will submit any updated serious adverse event data to the sponsor within 24 hours of it being available.

Since the criteria for the CTCAE grading scale differs from the regulatory criteria for serious adverse events, if adverse events correspond to grade 4 CTCAE toxicity grading scale criteria (e.g., laboratory abnormality reported as grade 4 without manifestation of life-threatening status), it will be left to the investigator's judgment to also report these abnormalities as serious adverse events. For any adverse event that applies to this situation, comprehensive documentation of the event's severity must be recorded in the subject medical records.

If the investigator becomes aware of serious adverse events suspected to be related to investigational product or any fatal adverse event (regardless of causality) after the protocol-required reporting period is complete, then these serious adverse events will be reported within 24 hours following the investigator's awareness of the event.

Serious adverse events reported outside of the protocol-required reporting period will be captured within the safety database as clinical trial cases and handled accordingly based on relationship to investigational product.

If further safety related data is needed to fulfill any regulatory reporting requirements for a reportable event, then additional information may need to be collected from the subject's records after the subject ends the study.

After the initial adverse event/serious adverse event report, the investigator is required to proactively follow each subject at subsequent visits/contacts. All adverse events and serious adverse events will be followed until resolution, stabilization, until the event is otherwise explained, or the subject is lost to follow-up.

Prompt notification by the investigator to the sponsor of serious adverse events is essential so that legal obligations and ethical responsibilities towards the safety of subjects and the safety of a study treatment under clinical investigation are met.

The sponsor has a legal responsibility to notify both the local regulatory authority and other regulatory agencies about the safety of a study treatment under clinical investigation. The sponsor will comply with country-specific regulatory requirements relating to safety reporting to the regulatory authority, IRBs/IECs, and investigators.

Individual safety reports must be prepared for suspected unexpected serious adverse reactions according to local regulatory requirements and sponsor policy and forwarded to investigators as necessary.

An investigator who receives an individual safety report describing a serious adverse event or other specific safety information (e.g., summary or listing of serious adverse events) from the sponsor will file it along with the Investigator's Brochure and will notify the IRB/IEC, if appropriate according to local requirements.

For studies in which the treatment assignment is blinded, to comply with worldwide reporting regulations for serious adverse events, the treatment assignment of subjects who develop serious, unexpected, and related adverse events may be unblinded by Amgen before submission to regulatory authorities. Aggregate analyses may also be unblinded by the Safety Assessment Team, as appropriate. Investigators will receive notification of related serious adverse events reports sent to regulatory authorities in accordance with local requirements.

### Adverse Events of Special Interest

Selected adverse events known as bemarituzumab Adverse Events of Special Interest are ocular adverse events of any grade or seriousness occurring up to 100 days after the last dose of bemarituzumab, and should be collected as adverse events. Ocular adverse events (including corneal adverse events) should be graded using an Ocular Toxicity Grading Scale Subjects should be assessed for possible bemarituzumab Adverse Events of Special Interest prior to each dose.

### Pregnancy and Lactation

Details of all pregnancies and/or lactation in female subjects and female partners of male subjects will be collected and reported. Abnormal pregnancy outcomes (e.g., spontaneous abortion, fetal death, stillbirth, congenital anomalies, and ectopic pregnancy) are considered serious adverse events.

A highly sensitive (serum) pregnancy test should be completed at screening and within 72 hours of initiation of investigational product for females of childbearing potential. Females who have undergone a bilateral tubal ligation/occlusion should have pregnancy testing per protocol requirements.

A highly sensitive (urine or serum) pregnancy test should be performed every cycle until 28 (+3) days after discontinuing protocol-required therapies. Additional on-treatment pregnancy testing may be performed at the investigator's discretion or as required per local laws and regulations.

### Pharmacokinetic Assessments

Whole blood samples of approximately 4 mL are collected for measurement of serum concentrations of bemarituzumab as specified in the Schedule of Activities (Table 3). The actual sample collection date and time (24-hour clock time) of each sample will be recorded.

### Pharmacogenetic Assessments

If the subject consents to the optional pharmacogenetic portion of this study, DNA analyses may be performed. These optional pharmacogenetic analyses focus on inherited genetic variations to evaluate their possible correlation to the disease and/or responsiveness to the therapies used in this study. The goals of the optional studies include the use of genetic markers to help in the investigation of cancer and/or to identify subjects who may have positive or negative response to investigational product. No additional samples are collected for this part of the study. For subjects who consent to this/these analysis/analyses, DNA may be extracted.

### Antibody Testing Procedures

Blood sample(s) for antibody testing are to be collected according to the time points specified in the Schedule of Activities (Table 3) for the measurement of anti-bemarituzumab antibodies. Samples testing positive for binding antibodies may be further characterized.

Subjects who test positive for antibodies at the final scheduled antibody time point and have clinical sequelae that are considered potentially related to an anti-bemarituzumab antibody response will also be asked to return for additional follow-up testing. This testing is to occur approximately every 3 months from the final scheduled antibody time point and continue until: (1) antibodies are no longer detectable; or (2) the subject has been followed for a period of at least 1 year (±4 weeks) post administration of bemarituzumab. All follow-up results, both positive and negative will be communicated to the sites. More frequent testing or testing for a longer period of time may be requested in the event of safety-related concerns.

### Biomarkers

Biomarkers are objectively measured and evaluated indicators of normal biologic processes, pathogenic processes, or pharmacologic responses to a therapeutic intervention.

### Biomarker Assessment to Determine Eligibility

Provision of archival tumor biopsy/resection or fresh biopsy that is formalin-fixed and embedded in paraffin is required for pre-screening FGFR2b overexpression testing by IHC. Fine needle aspirates, bone biopsies, and cytology samples are insufficient sample types. Primary tumor and metastatic sites of specimen collection are allowed. Fibroblast growth factor receptor 2b (FGFR2b) positive overexpression status is required for enrollment and subjects must consent to tumor tissue analysis. If fresh tissue samples need to be collected, these samples should be obtained following local standard of care procedures that are not expected to present any additional significant risk to the health, safety, and welfare of the subject.

Subjects are selected for enrollment based on FGFR2b overexpression, as determined by an investigational in vitro diagnostic (IVD) IHC assay at a central laboratory. The investigational IVD that is used to select subjects is the VENTANA FGFR2b (FPR2-D) Assay, an IHC test to detect the FGFR2b protein in neoplastic tissue. Subject tissue must be evaluated according to the VENTANA FGFR2b (FPR2-D) Assay scoring algorithm in order to determine FGFR2b status. Timing of sample collection is described in the Schedule of Activities (Table 3).

Subjects who do not demonstrate FGFR2b overexpression using IHC will not be eligible for enrollment. Samples that result in FGFR2b overexpression exhibit any moderate to strong membrane staining. It is the responsibility of each investigator to obtain an adequate tumor specimen for analysis of FGFR2b positivity for enrollment. Standard of care biopsy specimens can be used if specimen was obtained within 6 months prior to pre-screening or fresh biopsy. Blocks are preferred, if available, but in lieu of blocks, unstained slides are recommended. The tumor specimen submitted should be of sufficient quantity to allow for IHC analysis; see the Laboratory Manual for details. Tumor slide or tumor block specimen processing, labeling, and shipping instructions are detailed in the Laboratory Manual that will be distributed with the specimen collection kit.

Once tumor specimens are received, analysis will be performed as efficiently as possible, and FGFR2b overexpression status results will be communicated back to the investigator or designee.

### Biomarker Assessment During the Study

PD-L1 testing will be performed on leftover archival pre-treatment tissue, if available, in the indications listed in the Schedule of Activities of Table 3.

### Biomarker Discovery

Samples will be collected for exploratory biomarker analysis, with the purpose to understand mechanism of action of the drug, assess the amount of tumor growth, or predict disease progression and responses to investigational product(s) or protocol-required therapies. These samples may also be used for developing methods that enable better understanding of the disease. Test(s) may be designed to identify subjects most likely to respond positively or negatively to bemarituzumab or protocol-required therapies to investigate and further understand gastric cancer.

Blood and tissue are collected for biomarker development at the time points specified in the Schedule of Activities (Table 3), if allowed according to local regulations and agreed by Ethic Committees (EC)/Institutional Review Board (IRB). Blood samples will be collected for assessment of circulating tumor/cell-free DNA mutational profiles for potential association with clinical endpoints. The circulating tumor/cell-free DNA assessments are used for profiling of somatic mutations. Tumor samples will be used to examine protein expression, including FGFR2b, PD-L1, and other potential biomarkers that may include RNA or DNA gene expression or somatic (tumor) mutation analysis.

### Statistical Considerations

### Statistical Hypotheses

No statistical hypothesis will be tested.

### Sample Size Determination

The probability of observing at least 1 DLT if the true DLT rate is 30% with 3 subjects treated at dose level in a given cohort is 66%; with 6 subjects the probability increases to 89%; with 13 or more subjects the probability is over 99%.

### Populations for Analysis

The following populations are defined in Table 11:

TABLE 11

| Population | Description |
|---|---|
| Enrolled | All enrolled subjects |
| Safety | All subjects who received at least 1 dose of bemarituzumab. |
| DLT | All subjects that are enrolled in dose exploration who received |
| | at least one dose of bemarituzumab and planned backbone treatment |
| | (CAPOX, CAPOX + nivolumab, SOX or SOX + nivolomab); and met |
| | either of the following: 1) experienced a DLT; or 2) on-study |
| | 21 days following first dose of Investigational product and |
| | received 100% of the planned IV dosed agents and received |
| | at least 80% of the planned orally dosed agents. |

### Covariates

The relationship between covariates and endpoints may be explored and specified in the statistical analysis plan if appropriate.

### Subgroups

Subgroups may be explored and specified in the statistical analysis plan if appropriate.

### Statistical Analyses

The statistical analysis plan will be developed and finalized before database lock. Below is a summary of the timing and methods for the planned statistical analyses.

### Planned Analyses

### Interim Analysis and Early Stopping Guidelines

During DLRMs, all available cumulative data (safety, tolerability, laboratory, PK, and efficacy) will be reviewed by cohort prior to making dose escalation or dose de-escalation recommendations. Adverse events and DLTs observed in all subjects will be evaluated continually and fully integrated into all DLRMs and considered in all enrollment and dosing decisions. A DLRM will occur during dose exploration and 21 days following last subject enrolled in a dose cohort.

The guidelines for dose escalation or de-escalation to the next dose level are determined by using a BOIN design (see Table 6).

The primary analysis is planned after all subjects complete the SFU period. The final analysis is planned after all subjects have ended the study.

### Methods of Analyses

Data will be summarized by cohort and dose level. Descriptive statistics on continuous data will include means, medians, standard deviations and ranges, while categorical data will be summarized using frequency counts and percentages. Unless otherwise specified, data will be summarized using the Safety Analysis Set.

Confidence intervals (CI) for proportions will be estimated using an exact method proposed by Clopper-Pearson (Clopper and Pearson, 1934). Kaplan-Meier (KM) methods will be used to estimate the median and percentiles for time to event endpoints with CI calculated by using the Brookmeyer and Crowley method (Brookmeyer and Crowley, 1982). Kaplan-Meier methods will be used to estimate landmarks for time to event endpoints (e.g., 1-year OS) with the Greenwood formula (Kalbfleisch and Prentice, 1980) used to estimate the standard error in the CI calculation.

### Efficacy Analyses

| Endpoint/Estimand | Statistical Analysis Methods |
|---|---|
| Primary | Not applicable |
| Secondary | Listings of secondary efficacy endpoints will be produced for all |
| | subjects. |
| | The proportion of subjects with an objective response will be |
| | estimated along with a 95% CI. The same method will be used to |
| | summarize disease control rate. |
| | Kaplan-Meier (KM) quartiles with 95% CIs, and KM estimates with |
| | 95% CIs at landmark time points will be estimated for the following |
| | endpoints: duration of objective response (for only responding |
| | subjects), PFS, and OS. |

### Safety Analyses

| Endpoint | Statistical Analysis Methods |
|---|---|
| Primary | The incidence of DLTs, Treatment emergent |
| | adverse events (TEAEs), clinical |
| | laboratory abnormalities, vital signs, and |
| | corneal findings will be tabulated. |

### Adverse Events

Subject incidence of all treatment-emergent adverse events will be tabulated by system organ class and preferred term. Tables of grade ≥3 events, fatal adverse events, serious adverse events, adverse events leading to interruption/withdrawal from investigational product, and adverse events of interest will also be provided.

### Laboratory Test Results

The analyses of safety laboratory endpoints will include summary statistics and shifts in grades of safety laboratory values between baseline and the worst on-study value.

### Vital Signs

The analyses of vital signs will include summary statistics and shifts in vital sign values between baseline and the worst on-study value.

### Physical Measurements

The analyses of physical measurements will include summary statistics.

### Electrocardiogram

The ECG measurements from this clinical study are performed as per standard of care for routine safety monitoring, rather than for purposes of assessment of potential QTc effect. Since these evaluations may not necessarily be performed under the rigorous conditions expected to lead to meaningful evaluation of QTc data; summaries and statistical analyses of ECG measurements are not planned, and these data would not be expected to be useful for meta-analysis with data from other trials.

### Antibody Formation

The incidence and percentage of subjects who develop anti-bemarituzumab antibodies at any time will be tabulated by treatment group.

### Exposure to Investigational Product

The number of days on investigational product, the total dose of investigational product, and dose intensity will be summarized using descriptive statistics.

### Exposure to Non-Investigational Product

The number of days on non-investigational product, the total dose of non-investigational product, and dose intensity will be summarized using descriptive statistics.

### Exposure to Concomitant Medication

Number and proportion of subjects receiving therapies of interest will be summarized by preferred term for each treatment group as coded by the World Health Organization Drug dictionary.

### Other Analyses

Pharmacokinetic parameters for bemarituzumab including, but not limited to, AUC, Cₘₐₓ, C_{trough} will be determined. PK data collected from this study in combination with PK data collected from other bemarituzumab studies will be used for population PK analysis. Additional analyses will be performed to evaluate relationships between bemarituzumab exposure and selected safety or efficacy or any relevant biomarker endpoints if data are available. Details and results of these exploratory analyses will be described in separate reports.

### Example 2: Treatment of Gastric Cancer Patients with Bemarituzumab and CAPOX

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

Additionally, these patients are treated with CAPOX after the end of bemarituzumab infusion. Specifically, oxaliplatin administration commences on day 1 of each cycle at a dose of 130 mg/m² intravenously over 120 minutes. On days 1 through day 14 of each cycle, capecitabine is administered at a dose of 1000 mg/m² orally twice daily (i.e., 1000 mg/m² in the morning and 1000 mg/m² in the evening).

The combination of bemarituzumab and CAPOX is efficacious in treating the gastric or gastroesophageal junction adenocarcinoma in these patients as measured by objective response (defined as complete response (CR)+partial response (PR)), as determined per Response Evaluation Criteria in Solid Tumors [RECIST v1.1]).

### Example 3: Treatment of Gastric Cancer Patients with Bemarituzumab and SOX

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

Additionally, these patients are treated with SOX after the end of bemarituzumab infusion. Specifically, oxaliplatin administration commences on day 1 of each cycle at a dose of 130 mg/m² intravenously over 120 minutes. On days 1 through day 14 of each cycle, S-1 is administered at a dose of 40 mg/m² orally twice daily (i.e., 40 mg/m² in the morning and 40 mg/m² in the evening).

The combination of bemarituzumab and SOX is efficacious in treating the gastric or gastroesophageal junction adenocarcinoma in these patients as measured by objective response (defined as complete response (CR)+partial response (PR)), as determined per Response Evaluation Criteria in Solid Tumors [RECIST v1.1]).

### Example 4: Treatment of Gastric Cancer Patients with Bemarituzumab and CAPOX

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

Additionally, these patients are treated with CAPOX after the end of bemarituzumab infusion. Specifically, oxaliplatin administration commences on day 1 of each cycle at a dose of 130 mg/m² intravenously over 120 minutes. On days 1 through day 14 of each cycle, capecitabine is administered at a dose of 1000 mg/m² orally twice daily (i.e., 1000 mg/m² in the morning and 1000 mg/m² in the evening).

The combination of bemarituzumab and CAPOX is efficacious in treating the gastric or gastroesophageal junction adenocarcinoma in these patients as measured by progression free survival and/or overall survival.

### Example 5: Treatment of Gastric Cancer Patients with Bemarituzumab and SOX

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

Additionally, these patients are treated with SOX after the end of bemarituzumab infusion. Specifically, oxaliplatin administration commences on day 1 of each cycle at a dose of 130 mg/m² intravenously over 120 minutes. On days 1 through day 14 of each cycle, S-1 is administered at a dose of 40 mg/m² orally twice daily (i.e., 40 mg/m² in the morning and 40 mg/m² in the evening).

The combination of bemarituzumab and SOX is efficacious in treating the gastric or gastroesophageal junction adenocarcinoma in these patients as measured by progression free survival and/or overall survival.

### Example 6: Treatment of Gastric Cancer Patients with Bemarituzumab, CAPOX, and Nivolumab

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

These patients are also treated with nivolumab after the end of bemarituzumab infusion. Nivolumab is administered on day 1 of each cycle at a dose of 360 mg intravenously over 30 minutes (±10 minutes).

Additionally, these patients are treated with CAPOX after the end of nivolumab infusion. Specifically, oxaliplatin administration commences on day 1 of each cycle at a dose of 130 mg/m² intravenously over 120 minutes. On days 1 through day 14 of each cycle, capecitabine is administered at a dose of 1000 mg/m² orally twice daily (i.e., 1000 mg/m² in the morning and 1000 mg/m² in the evening).

The combination of bemarituzumab, CAPOX, and nivolumab is efficacious in treating the gastric or gastroesophageal junction adenocarcinoma in these patients as measured by objective response (defined as complete response (CR)+partial response (PR)), as determined per Response Evaluation Criteria in Solid Tumors [RECIST v1.1]).

### Example 7: Treatment of Gastric Cancer Patients with Bemarituzumab, SOX, and Nivolumab

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

These patients are also treated with nivolumab after the end of bemarituzumab infusion. Nivolumab is administered on day 1 of each cycle at a dose of 360 mg intravenously over 30 minutes (±10 minutes)

Additionally, these patients are treated with SOX after the end of nivolumab infusion. Specifically, oxaliplatin administration commences on day 1 of each cycle at a dose of 130 mg/m² intravenously over 120 minutes. On days 1 through day 14 of each cycle, S-1 is administered at a dose of 40 mg/m² orally twice daily (i.e., 40 mg/m² in the morning and 40 mg/m² in the evening).

The combination of bemarituzumab, SOX, and nivolumab is efficacious in treating the gastric or gastroesophageal junction adenocarcinoma in these patients as measured by objective response (defined as complete response (CR)+partial response (PR)), as determined per Response Evaluation Criteria in Solid Tumors [RECIST v1.1]).

### Example 8: Treatment of Gastric Cancer Patients with Bemarituzumab, CAPOX, and Nivolumab

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

These patients are also treated with nivolumab after the end of bemarituzumab infusion. Nivolumab is administered on day 1 of each cycle at a dose of 360 mg intravenously over 30 minutes (±10 minutes)

Additionally, these patients are treated with CAPOX after the end of nivolumab infusion. Specifically, oxaliplatin administration commences on day 1 of each cycle at a dose of 130 mg/m² intravenously over 120 minutes. On days 1 through day 14 of each cycle, capecitabine is administered at a dose of 1000 mg/m² orally twice daily (i.e., 1000 mg/m² in the morning and 1000 mg/m² in the evening).

The combination of bemarituzumab, CAPOX, and nivolumab is efficacious in treating the gastric or gastroesophageal junction adenocarcinoma in these patients as measured by progression free survival and/or overall survival.

### Example 9: Treatment of Gastric Cancer Patients with Bemarituzumab, SOX, and Nivolumab

A cohort of patients having pathologically confirmed locally advanced or metastatic gastric or gastroesophageal junction adenocarcinoma with FGFR2b overexpression is treated with bemarituzumab at one of the following doses:
(1) a dose of 15 mg/kg intravenously on cycle 1 day 1 (D1) and Q3W thereafter, and a single dose of 7.5 mg/kg on cycle 1, day 8; or
(2) a dose of 22 mg/kg intravenously on cycle 1 D1 and Q3W thereafter, and a single dose of 11 mg/kg on cycle 1, day 8.

These patients are also treated with nivolumab after the end of bemarituzumab infusion. Nivolumab is administered on day 1 of each cycle at a dose of 360 mg intravenously over 30 minutes (±10 minutes)

Additionally, these patients are treated with SOX after the end of nivolumab infusion. Specifically, oxaliplatin administration commences on day 1 of each cycle at a dose of 130 mg/m² intravenously over 120 minutes. On days 1 through day 14 of each cycle, S-1 is administered at a dose of 40 mg/m² orally twice daily (i.e., 40 mg/m² in the morning and 40 mg/m² in the evening).

The combination of bemarituzumab, SOX, and nivolumab is efficacious in treating the gastric or gastroesophageal junction adenocarcinoma in these patients as measured by progression free survival and/or overall survival.

### Example 10: Results from Phase 1b Study Evaluating the Safety, Tolerability, and Pharmacokinetics of Bemarituzumab in Combination with Other Anti-Cancer Therapies in Subjects with Previously Untreated Advanced Gastric or Gastroesophageal Junction Cancer

Three cohorts of patients having pathologically confirmed locally advanced or metastatic gastric or gastroesophageal junction adenocarcinoma with FGFR2b overexpression were administered the anti-FGFR2b antibody bemarituzumab (Bema) intravenously in combination with CAPOX with or without nivolumab (Nivo) according to the protocol described in Example 1. Specifically, two dose levels of bemarituzumab were tested in Cohort A as described in Example 1 (i.e., Cohort A, Dose Level 1 (DL1) and Cohort A DL2), along with Cohort C, according to the following dosing schedule:
Cohort A DL1: Bema 15 mg/kg IV Q3W+a single 7.5 mg/kg dose on day 8 of the first cycle (“C1D8”)+CAPOX (N=6; pharmacokinetic (PK) data available for 6/6 patients)
Cohort A DL2: Bema 22 mg/kg IV Q3W+a single 11 mg/kg dose C1D8+CAPOX (N=4; PK data available for 4/4 patients)
Cohort C: Bema 22 mg/kg IV Q3W+a single 11 mg/kg dose C1D8+CAPOX+Nivo (N=6; PK data available for 6/6 patients).

Bemarituzumab serum concentration was measured at pre-specified timepoints as shown in Table 3. The preliminary PK parameter summaries for bemarituzumab in treatment Cycles 1 and 2 are shown in Table 12.

TABLE 12
Summary of median Cₘₐₓ and C_{trough} for Cohort
A DL1&2, Cohort C, Cycle 1 and Cycle 2

| | | | Cycle 1 | Cycle 2 | Cycle 2 |
|---|---|---|---|---|---|
| | Dose | Cycle 1 Cₘₐₓ | C_{trough} | Cₘₐₓ | C_{trough} |
| Cohort | (mg/kg) | (μg/mL) | (μg/mL) | (μg/mL) | (μg/mL) |
| A DL1 | 15 Q3W + 7.5 | 360.8 | 111.5 | 457.0 | 86.6 |
| | C1D8 | | | | |
| A DL2 | 22 Q3W + 11 | 333.8 | 125.9 | 655.8 | 71.1 |
| | C1D8 | | | | |
| C | 22 Q3W + 11 | 381.4 | 130.1 | 549.8 | 133.0 |
| | C1D8 (w/Nivo) | | | | |

No drug-drug interaction (DDI) with respect to PK was observed between bemarituzumab and nivolumab in a CAPOX background. In addition, there were no new safety findings in the patients of cohort A (DL1 & DL2), and cohort C based on the known bemarituzumab safety profile.

### REFERENCES

Each of the following references is incorporated by reference in its entirety herein.
International Committee of Medical Journal Editors, Uniform Requirements for Manuscripts Submitted to Biomedical Journals: Writing and Editing for Biomedical Publication. 2019. http://www.icmje.org/
Ahn S, Lee J, Hong M, et al. FGFR2 in gastric cancer: protein overexpression predicts gene amplification and high H-index predicts poor survival. Mod Pathol. 2016; 29(9):1095-1103.
Al-Batran S E, Hartmann J T, Hofheinz R, et al. Biweekly Fluorouracil, Leucovorin, Oxaliplation and Docetaxel (FLOT) for Patients with Metastatic Adenocarcinoma of the Stomach or Esophagogastric Junction: A Phase II Trial of the Arbeitsgemeinschaft Internistische Onkologie. Annals of Oncology. 2008; 19:1882-1887.
Andre F, Ranson M, Dean E, et al. Abstract LB-145: Results of a Phase I Study of AZD4547, an Inhibitor of Fibroblast Growth Factor Receptor (FGFR), in Patients with Advanced Solid Tumors. AACR 104th Annual Meeting 2013, Washington, DC, American Association of Cancer Research.
Bang Y J, Van Cutsem E, Feyereislova A, et al. Trastuzumab in combination with chemotherapy versus chemotherapy alone for treatment of HER2-positive advanced gastric or gastro-oesophageal junction cancer (ToGA): a phase 3, open-label, randomised controlled trial [published correction appears in Lancet. 2010 Oct. 16; 376(9749):1302]. Lancet. 2010; 376(9742):687-697.
Bray F, Ferlay J, Soerjomataram I, Siegel R L, Torre L A, Jemal A. Global cancer statistics 2018: GLOBOCAN estimates of incidence and mortality worldwide for 36 cancers in 185 countries. CA Cancer J Clin. 2018 November; 68(6):394-424.
Blum M, Suzuki A, Ajani J A. A comprehensive review of S-1 in the treatment of advanced gastric adenocarcinoma. Future Oncology. 2011; 7(6).
Boku N, Ryu M-H, Kato K, et al. Safety and efficacy of nivolumab in combination with S-1/capecitabine plus oxaliplatin in patients with previously untreated, unresectable, advanced, or recurrent gastric/gastroesophageal junction cancer: interim results of a randomized, phase II trial (ATTRACTION-4). Ann Oncol. 2019; 30(2):250-258.
Boku N, Yamamoto S, Fukuda S, et al. Fluorouracil versus combination or irinotecan plus cisplatin versus S-1 in metastatic gastric cancer: a randomized phase 3 study. The Lancet. 2009; 10(11):P1063-P1069.
Brookmeyer, R. and Crowley, J. A confidence interval for the median survival time. Biometrics. 1982; 38:29-41.
Brown A P, Courtney C L, King L M, et al. Cartilage Dysplasia and Tissue Mineralization in the Rat Following Administration of a FGF Receptor Tyrosine Kinase Inhibitor. Toxicologic Pathology. 2005; 33:449-455.
Catenacci D V T, Rasco D, Lee J, et al. Phase I Escalation and Expansion Study of Bemarituzumab (FPA144) in Patients with Advanced Solid Tumors and FGFR2b Selected Gastroesophageal Adenocarcinoma. J Clin Oncol. 2020; 82(21):2418-2426.
Clopper, C J and Pearson E S. The use of confidence or fiducial limits illustrated in the case of the binomial. Biometrika. 1934:404-416.
Dienstmann R, Bahleda R, Barbara A, et al. First in human study of JNJ-42756493, a potent pan fibroblast growth factor receptor (FGFR) inhibitor in patients with advanced solid tumors. AACR 105th Annual Meeting 2014, San Diego, C A.
Eisenhauer E A, Therasse P, Bogaerts J, et al. New response evaluation criteria in solid tumours: revised RECIST guideline (version 1.1). Eur J Cancer. 2009; 45(2):228-247.
Fuchs C, Tomasek J, Yong C, et al. Ramucirumab Monotherapy for Previously Treated Advanced Gastric or Gastro-Oesophageal Junction Adenocarcinoma (REGARD): an International, Randomised, Multicentre, Placebo-Controlled, Phase 3 Trial. Lancet Oncology. 2014; 383:31-39.
GASTRIC (Global Advanced/Adjuvant Stomach Tumor Research International Collaboration) Group, Paoletti X, Oba K, et al. Benefit of adjuvant chemotherapy for resectable gastric cancer: a meta-analysis. JAMA. 2010; 303(17):1729-1737.
Gemo A, Deshpande A, Palencia S, et al. FPA144: A Therapeutic Antibody for Treating Ptients with Gastric Cancers Bearing FGFR2 Gene Amplification. AACR 105th Annual Meeting 2014, San Diego California.
Janjigian Y Y, Shitara K, Moehler M, et al. First-line nivolumab plus chemotherapy versus chemotherapy alone for advanced gastric, gastro-oesophageal junction, and oesophageal adenocarcinoma (CheckMate 649): a randomized, open-label, phase 3 trial. Lancet. 2021; 398(10294):27-40.
Jung E J, Jung E J, Min S Y, Kim M A, Kim W H. Fibroblast growth factor receptor 2 gene amplification status and its clinicopathologic significance in gastric carcinoma. Hum Pathol. 2012; 43(10):1559-1566.
Kalbfleisch, J. D. and Prentice, R. L. (1980) The statistical analysis of failure time data. John Wiley and Sons, New York.
Kang Y K, Kang W K, Shin D B, et al. Capecitabine/Cisplatin Versus 5 Fluorouracil/Cisplatin as First-Line Therapy in Patients with Advanced Gastric Cancer: A Randomised Phase III Noninferiority Trial. Annals of Oncology. 2009; 20:666-673.
Katoh Y and Katoh M. Hedgehog signaling pathway and gastrointestinal stem cell signaling network (review). Int J Mol Med. 2006; 18(6):1019-1023.
Kim G M, Jeung H-C, Rha S Y, et al. A randomized phase III trial of S-1-oxaliplatin versus cepecitabine-oxaliplatin in advanced gastric cancer. Eur J Cancer. 2012; 48(4):518-526.
Keam B, Im S A, Han S W, et al. Modified FOLFOX-6 chemotherapy in advanced gastric cancer: Results of phase II study and comprehensive analysis of polymorphisms as a predictive and prognostic marker. BMC Cancer. 2008; 8:148.
Kim H S, Kim J H, Jang H J, Han B, Zang D Y. Pathological and Prognostic Impacts of FGFR2 Overexpression in Gastric Cancer: A Meta-Analysis. J Cancer. 2019; 10(1):20-27. Published 2019 Jan. 1.
Matsumoto K, Arao T, Hamaguchi T, et al. FGFR2 gene amplification and clinicopathological features in gastric cancer. Br J Cancer. 2012; 106(4):727-732.
Moehler M, Shitara K, Garrido M, et al. LBA6_PR Nivolumab (nivo) plus chemotherapy (chemo) versus chemo as first-line (1L) treatment for advanced gastric cancer/gastroesophageal junction cancer (GC/GEJC)/esophageal adenocarcinoma (EAC): First results of the CheckMate 649 study. Ann Oncol. 2020; 31:S1191 10.1016/j.annonc.2020.08.2296.
National Cancer Institute (2015). SEER Stat Fact Sheets: Stomach Cancer. Retrieved Feb. 25, 2016, from http://seer.cancer.gov/statfacts/html/stomach.html.
National Comprehensive Cancer Network, NCCN Guidelines for Gastric Cancer 2021. https://www.nccn.org/guidelines/guidelines-detail?category=1&id=1434.
Okines A F C, Norman A R, McCloud P, Kang Y K, Cunningham D. Meta-analysis of the REAL-2 and ML17032 trials: evaluating capecitabine-based combination chemotherapy and infused 5-fluorouracil-based combination chemotherapy for the treatment of advanced oesophago-gastric cancer. Ann Oncol. 2009; 20(9):1529 1534.
Park Y H, Lee J-L, Ryoo B-Y, et al. Capecitabine in combination with oxaliplatin (XELOX) as a first-line therapy for advanced gastric cancer. Cancer Chemother Pharmacol. 2008; 61(4):623-629.
Sequist L V, Cassier P, Varga A, et al. Phase I Study of BGJ398, a Selective Pan-FGFR Inhibitor in Genetically Preselected Advanced Solid Tumors. AACR 2014 Annual Meeting, San Diego, C A.
Shah M A, Kang Y K, Thuss-Patience P C, et al. Biomarker analysis of the GATSBY study of trastuzumab emtansine versus a taxane in previously treated HER2 positive advanced gastric/gastroesophageal junction cancer. Gastric Cancer. 2019; 22(4):803-816.
Shinkawa T, Nakamura K, Yamane N, et al. The Absence of Fucose but Not the Presence of Galactose or Bisecting N-Acetylglucosamine of Human IgG1 Complex-type Oligosaccharides Shows the Critical Role of Enhancing Antibody-dependent Cellular Cytotoxicity. J Biol Chem. 2003; 278(5):3466-3473.
Shitara K, Doi T, Dvorkin M, et al. Trifluridine/tipiracil versus placebo in patients with heavily pretreated metastatic gastric cancer (TAGS): a randomised, double-blind, placebo-controlled, phase 3 trial. Lancet Oncol. 2018; 19(11):1437-1448.
The GASTRIC Group. Benefit of Adjuvant Chemotherapy for Resectable Gastric Cancer. JAMA. 2010; 303(1729-1737):1729.
Tokunaga R, Imamura Y, Nakamura K, et al. Fibroblast growth factor receptor 2 expression, but not its genetic amplification, is associated with tumor growth and worse survival in esophagogastric junction adenocarcinoma. Oncotarget. 2016; 7(15):19748-19761.
Waddell T, Verheij M, Allum W, et al. Gastric Cancer: ESMO-ESSO-ESTRO Clinical Practice Guidelines for Diagnosis, Treatment, and Follow-up. Radiotherapy and Oncology. 2014; 110:189-194.
Wagner A D, Grothe W, Haerting J, Kleber G, Grothey A, Fleig W E. Chemotherapy in advanced gastric cancer: a systematic review and meta-analysis based on aggregate data. J Clin Oncol. 2006; 24(18):2903-2909.
Wagner A D, Syn N L, Moehler M, et al. Chemotherapy for advanced gastric cancer. Cochraine Database Syst Rev. 2017; 8(8):CD004064.
Yamada Y, Higuchi K, Nishikawa K, et al. Phase III Study comparing oxaliplatin plus S-1 with cisplatin plus S-1 in chemotherapy-naïve patients with advanced gastric cancer. Ann Oncol. 2014; 26(1):141-148.

All references, including publications, patent applications, and patents, cited herein are hereby incorporated by reference to the same extent as if each reference were individually and specifically indicated to be incorporated by reference and were set forth in its entirety herein.

The use of the terms “a” and “an” and “the” and “at least one” and similar referents in the context of describing the invention (especially in the context of the following claims) are to be construed to cover both the singular and the plural, unless otherwise indicated herein or clearly contradicted by context. The use of the term “at least one” followed by a list of one or more items (for example, “at least one of A and B”) is to be construed to mean one item selected from the listed items (A or B) or any combination of two or more of the listed items (A and B), unless otherwise indicated herein or clearly contradicted by context. The terms “comprising,” “having,” “including,” and “containing” are to be construed as open-ended terms (i.e., meaning “including, but not limited to,”) unless otherwise noted. Recitation of ranges of values herein are merely intended to serve as a shorthand method of referring individually to each separate value falling within the range, unless otherwise indicated herein, and each separate value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein, is intended merely to better illuminate the invention and does not pose a limitation on the scope of the invention unless otherwise claimed. No language in the specification should be construed as indicating any non-claimed element as essential to the practice of the invention.

Preferred embodiments of this invention are described herein, including the best mode known to the inventors for carrying out the invention. Variations of those preferred embodiments may become apparent to those of ordinary skill in the art upon reading the foregoing description. The inventors expect skilled artisans to employ such variations as appropriate, and the inventors intend for the invention to be practiced otherwise than as specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

1. A method of treating gastric cancer in a subject, comprising:
administering to the subject an anti-FGFR2b antibody; and
administering to the subject either
(a) oxaliplatin and capecitabine; or
(b) oxaliplatin and S-1.

2. The method of claim 1, wherein at least 15 mg/kg of the anti-FGFR2b antibody is administered Q3W to the subject.

3. The method of claim 1, wherein greater than 15 mg/kg to no more than 22 mg/kg of the anti-FGFR2b antibody is administered Q3W to the subject.

4. The method of claim 2, further comprising administering an additional 5-15 mg/kg of the anti-FGFR2b antibody 7-10 days after a first administration of the Q3W administration of the anti-FGFR2b antibody.

5. The method of claim 4, wherein an additional 7-8 mg/kg of the anti-FGFR2b antibody is administered to the subject.

6. The method of claim 1, wherein at least 20 mg/kg of the anti-FGFR2b antibody is administered Q3W to the subject.

7. The method of claim 6, wherein 20-30 mg/kg of the anti-FGFR2b antibody is administered Q3W to the subject.

8. The method of claim 6, further comprising administering an additional 10-15 mg/kg of the anti-FGFR2b antibody 7-10 days after a first administration of the Q3W administration of the anti-FGFR2b antibody.

9. The method of claim 8, wherein an additional 9-13 mg/kg or 10-12 mg/kg of the anti-FGFR2b antibody is administered to the subject.

10. The method of claim 1, wherein the anti-FGFR2b antibody is administered intravenously.

11. The method of claim 1, comprising administering to the subject at least 65 mg/m² of the oxaliplatin intravenously Q3W and administering at least 500 mg/m² of the capecitabine orally twice a day.

12. The method of claim 11, wherein 100-150 mg/m² of the oxaliplatin is administered intravenously Q3W to the subject, and 750-1500 mg/m² of the capecitabine is administered orally twice a day.

13. The method of claim 10, comprising administering at least 65 mg/m² of the oxaliplatin intravenously Q3W to the subject, and administering at least 20 mg/m² of the S-1 orally twice a day.

14. The method of claim 13, wherein 100-150 mg/m² of the oxaliplatin is administered intravenously Q3W to the subject, and 30-60 mg/m² of the S1 is administered orally twice a day.

15. The method of claim 1,
wherein the anti-FGFR2b antibody comprises the heavy chain of SEQ ID NO: 1 and the light chain of SEQ ID NO: 2, and wherein the anti-FGFR2b antibody is afucosylated,
wherein 15-22 mg/kg of the anti-FGFR2b antibody is administered intravenously Q3W to the subject, and an additional 7-12 mg/kg of the anti-FGFR2b antibody is further administered 7-10 days after a first administration of the anti-FGFR2b antibody Q3W, and
wherein:
(a) 100-150 mg/m² of the oxaliplatin is administered intravenously Q3W to the subject and 750-1500 mg/m² of the capecitabine is administered orally twice a day; or
(b) 10-150 mg/m² of the oxaliplatin is administered intravenously Q3W to the subject and 30-60 mg/m² of the S-1 is administered orally twice a day.

16. The method of claim 1,
wherein the anti-FGFR2b antibody comprises the heavy chain of SEQ ID NO: 1, and the light chain of SEQ ID NO: 2 and is afucosylated, and
wherein 15-22 mg/kg of the anti-FGFR2b antibody is administered intravenously Q3W to the subject, and an additional 7-12 mg/kg of the anti-FGFR2b antibody is further administered 7-10 days after a first administration of the anti-FGFR2b antibody in the Q3W regimen, and
wherein:
(a) 130 mg/m² of the oxaliplatin is administered intravenously Q3W to the subject and 1000 mg/m² of the capecitabine is administered orally twice a day; or
(b) 130 mg/m² of the oxaliplatin is administered intravenously Q3W to the subject and 40 mg/m² of the S-1 is administered orally twice a day.

17. The method of claim 1,
wherein the anti-FGFR2b antibody comprises the heavy chain of SEQ ID NO: 1, and the light chain of SEQ ID NO: 2, and wherein the anti-FGFR2b antibody is afucosylated, and
wherein 22-30 mg/kg of the anti-FGFR2b antibody is administered intravenously Q3W to the subject, and an additional 10-12 mg/kg of the anti-FGFR2b antibody is further administered 7-10 days after a first administration of the anti-FGFR2b antibody Q3W, and
wherein:
(a) 130 mg/m² of the oxaliplatin is administered intravenously Q3W to the subject and 1000 mg/m² of the capecitabine is administered orally twice a day; or
(b) 130 mg/m² of the oxaliplatin is administered intravenously Q3W to the subject and 40 mg/m² of the S-1 is administered orally twice a day.

18. The method of claim 1, further comprising administering an anti-PD-1 or anti PD-L1 antibody to the subject.

19. The method of claim 18, wherein the anti-PD-1 or anti PD-L1 antibody is administered Q3W.

20. The method of claim 18, wherein the anti-PD-1 antibody comprises nivolumab, pembrolizumab, orcemiplimab, or wherein the anti-PD-L1 antibody comprises atezolizumab, avelumab, or durvalumab.

21. The method of claim 16, further comprising administering at least 300 mg nivolumab to the subject Q3W.

22. The method of claim 21, wherein 300 mg-400 mg of the nivolumab is administered Q3W.

23. The method of claim 21, wherein 360 mg of the nivolumab is administered Q3W.

24. The method of claim 1, wherein the anti-FGFR2b antibody comprises:
a heavy chain variable region comprising a heavy chain complementarity determining region (HCDR) 1 of SEQ ID NO: 6, a HCDR2 of SEQ ID NO: 7, and a HCDR3 of SEQ ID NO: 8; and
a light chain variable region comprising a light chain complementarity determining region (LCDR) 1 of SEQ ID NO: 9, a LCDR2 of SEQ ID NO: 10, and a LCDR3 of SEQ ID NO: 11.

25. The method of claim 24, wherein the anti-FGFR2b antibody is afucosylated.

26. The method of claim 24, wherein the heavy chain variable region of the anti-FGFR2b antibody comprises an amino acid sequence at least 95% identical to SEQ ID NO: 4, and wherein the light chain variable region of the anti-FGFR2b antibody comprises an amino acid sequence at least 95% identical to SEQ ID NO: 5.

27. The method of claim 24, wherein the heavy chain variable region of the anti-FGFR2b antibody comprises the amino acid sequence of SEQ ID NO: 4, and wherein the light chain variable region of the anti-FGFR2b antibody comprises the amino acid sequence of SEQ ID NO: 5.

28. The method of claim 24, wherein the anti-FGFR2b antibody comprises the heavy chain of SEQ ID NO: 1, and the light chain of SEQ ID NO: 2.

29. The method of claim 1, wherein the anti-FGFR2b antibody is bemarituzumab.

30. The method of claim 1, wherein cells of the gastric cancer overexpress FGFR2b.

31. The method of claim 1, wherein the cells of the gastric cancer overexpress FGFR2b mRNA or protein, or comprise an FGFR2 gene amplification.

32. The method of 30, wherein cells of the gastric cancer overexpress FGFR2b as determined by immunohistochemistry (IHC).

33. The method of claim 30, wherein at least 10% of cells of the gastric cancer express FGFR2b as determined by IHC.

34. (canceled)

35. The method of claim 30, wherein any cells of the gastric cancer have a FGFR2b staining intensity of at least 2+, or 3+ as determinized by IHC.

36. The method of claim 30, wherein at least 10% of cells of the gastric cancer have a FGFR2b staining intensity of at least 2+, or 3+ as determinized by IHC.

37. The method of claim 1, which comprises administering (a) oxaliplatin and capecitabine to the subject.

38. The method of claim 1, wherein the gastric cancer comprises gastric cancer or gastroesophageal junction cancer.


SEQUENCE LISTING (12 sequences)

SEQ ID NO: 1 - protein, 801 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKHSGINSSNAEVLALFNVTEADAGEYICKVSNYIGQANQSAWLTVLPKQQAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT

SEQ ID NO: 2 - protein, 444 aa, Synthetic construct
QVQLVQSGAEVKKPGSSVKVSCKASGYIFTTYNVHWVRQAPGQGLEWIGSIYPDNGDTSYNQNFKGRATITADKSTSTAYMELSSLRSEDTAVYYCARGDFAYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 3 - protein, 214 aa, synthetic construct
DIQMTQSPSSLSASVGDRVTITCKASQGVSNDVAWYQQKPGKAPKLLIYSASYRYTGVPSRFSGSGSGTDFTFTISSLQPEDIATYYCQQHSTTPYTFGQGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 4 - protein, 114 aa, synthetic construct [sequence omitted]

SEQ ID NO: 5 - protein, 107 aa, synthetic construct [sequence omitted]

SEQ ID NO: 6 - protein, 5 aa, synthetic construct
TYNVH

SEQ ID NO: 7 - protein, 17 aa, synthetic construct
SIYPDNGDTSYNQNFKG

SEQ ID NO: 8 - protein, 5 aa, synthetic construct
GDFAY

SEQ ID NO: 9 - protein, 11 aa, synthetic construct
KASQGVSNDVA

SEQ ID NO: 10 - protein, 7 aa, synthetic construct
SASYRYT

SEQ ID NO: 11 - protein, 9 aa, synthetic construct
QQHSTTPYT

SEQ ID NO: 12 - protein, 800 aa, Homo sapiens
RPSFSLVEDTTLEPEEPPTKYQISQPEVYVAAPGESLEVRCLLKDAAVISWTKDGVHLGPNNRTVLIGEYLQIKGATPRDSGLYACTASRTVDSETWYFMVNVTDAISSGDDEDDTDGAEDFVSENSNNKRAPYWTNTEKMEKRLHAVPAANTVKFRCPAGGNPMPTMRWLKNGKEFKQEHRIGGYKVRNQHWSLIMESVVPSDKGNYTCVVENEYGSINHTYHLDVVERSPHRPILQAGLPANASTVVGGDVEFVCKVYSDAQPHIQWIKHVEKNGSKYGPDGLPYLKVLKAAGVNTTDKEIEVLYIRNVTFEDAGEYTCLAGNSIGISFHSAWLTVLPAPGREKEITASPDYLEIAIYCIGVFLIACMVVTVILCRMKNTTKKPDFSSQPAVHKLTKRIPLRRQVTVSAESSSSMNSNTPLVRITTRLSSTADTPMLAGVSEYELPEDPKWEFPRDKLTLGKPLGEGCFGQVVMAEAVGIDKDKPKEAVTVAVKMLKDDATEKDLSDLVSEMEMMKMIGKHKNIINLLGACTQDGPLYVIVEYASKGNLREYLRARRPPGMEYSYDINRVPEEQMTFKDLVSCTYQLARGMEYLASQKCIHRDLAARNVLVTENNVMKIADFGLARDINNIDYYKKTTNGRLPVKWMAPEALFDRVYTHQSDVWSFGVLMWEIFTLGGSPYPGIPVEELFKLLKEGHRMDKPANCTNELYMMMRDCWHAVPSQRPTFKQLVEDLDRILTLTTNEEYLDLSQPLEQYSPSYPDTRSSCSSGDDSVFSPDPMPYEPCLPQYPHINGSVKT